(19) United States
(12) Patent Application Publication
(10) Pub. No.: US 2019/0218613 A1
(43) Pub. Date: Jul. 18, 2019

(54) METHODS OF DETECTING THE FORMATION OF CELLULAR CLUSTERS OF RNA

(71) Applicant: The Regents of the University of California, Oakland, CA (US)

(72) Inventors: Ronald D. VALE, San Francisco, CA (US); Ankur JAIN, San Francisco, CA (US)

(73) Assignee: The Regents of the University of California, Oakland, CA (US)

(21) Appl. No.: 16/206,427

(22) Filed: Nov. 30, 2018

Related U.S. Application Data
(60) Provisional application No. 62/593,821, filed on Dec. 1, 2017.

(51) Int. Cl. C12Q 1/6883

(52) U.S. Cl. CPC C12Q 1/6883; C12Q 2600/158

(57) ABSTRACT

In one aspect, cells and cell-based assays for detecting the formation of cellular clusters of RNA (e.g., base-pairing mediated cellular clusters of RNA) are provided. In some embodiments, the cell comprises a heterologous polynucleotide comprising a promoter operably linked to a polynucleotide for encoding an RNA transcript comprising (i) an RNA sequence comprising a sequence that is prone to forming clusters of RNA and (ii) a binding motif for binding to a detectable molecule; and a heterologous detectable molecule that binds to the binding motif. In another aspect, methods of identifying an agent that dissolves or inhibits the formation of cellular clusters of RNA are provided.

### CROSS-REFERENCE TO RELATED APPLICATIONS

This application claims priority to U.S. Provisional Patent Application No. 62/593,821, filed Dec. 1, 2017, the entire contents of which are incorporated by reference herein.

### BACKGROUND OF THE INVENTION

Nucleotide repeat expansion disorders constitute some of the most common inherited diseases (see, e.g., Gatchel et al., Nat. Rev. Genet. 6:743, 2005 and La Spada and Taylor, Nat. Rev. Genet. 11:247, 2010). Several of the disease-associated repeat expansions comprise a nucleotide triplet of high G/C content, such as CAG in Huntington disease and spinocerebellar ataxias, and CTG in myotonic dystrophy (see, e.g., La Spada and Taylor, Nat. Rev. Genet. 11:247, 2010, and Krzyzosiak et al., Nucleic Acids Res. 40:11, 2012). Likewise, the expansion of the hexanucleotide GGGGCC in the C9orf72 gene is the most common mutation associated with familial amyotrophic lateral sclerosis (ALS) and frontotemporal dementia (FTD) (see, e.g., DeJesus-Hernandez et al., Neuron 72:245, 2011, and Renton et al., Neuron 72:257, 2011). A common pathological feature of these diseases is the accumulation of repeat containing transcripts into aberrant foci, and studies have suggested that nuclear foci are linked to cellular toxicity.

There remains a need for assays and methods for identifying agents that are useful in the treatment of diseases associated with repeat expansions or other diseases associated with RNA foci.

### BRIEF SUMMARY OF THE INVENTION

In a first aspect, the disclosure provides an isolated cell comprising: a heterologous polynucleotide comprising a promoter operably linked to a polynucleotide for encoding an RNA transcript comprising (i) an RNA sequence that is prone to forming clusters of RNA and (ii) a binding motif for binding to a detectable molecule; and a heterologous detectable molecule that binds to the binding motif In some embodiments, the RNA sequence comprises a sequence that is prone to forming clusters of RNA. In some embodiments, the formation of clusters of RNA is mediated by base pairing. In another aspect, the disclosure provides an isolated cell comprising clusters of RNA comprising an RNA transcript comprising (i) tandem nucleotide repeats and (ii) a binding motif for binding to a detectable molecule; and a heterologous detectable molecule that binds to the binding motif.

In some embodiments of this aspect, the sequence that is prone to forming clusters of RNA comprises tandem nucleotide repeats (e.g., multiple nucleotide repeats comprising at least 10, 15, 20, 25, 30, 40 or more adjacent repeated nucleotide sequences). In some embodiments, the tandem nucleotide repeats are trinucleotide repeats. The trinucleotide repeat sequences may be CAG repeats, CGG repeats, GCC repeats, GAA repeats, or CUG repeats. In particular embodiments, the RNA sequence comprises at least 30 repeats.

In some embodiments, the tandem nucleotide repeats are tetranucleotide repeats, pentanucleotide repeats, or hexanucleotide repeats. The tandem nucleotide repeat sequences may be GGGGCC repeats, CCUG repeats, or AUUCU repeats. In particular embodiments, the RNA sequence comprises at least 15 repeats.

In some embodiments, the tandem nucleotide repeats are contiguous (e.g., directly adjacent to each other) or non-contiguous (e.g., separated by 1 or more nucleotides).

In some embodiments, the binding motif comprises a hairpin loop sequence or an aptamer sequence. In some embodiments, the hairpin loop sequence comprises a plurality of hairpin loop nucleotide sequences separated by a spacer sequence.

In some embodiments of this aspect, the detectable molecule is a heterologous protein that comprises a detectable label. In some embodiments, the detectable label is a fluorophore.

In some embodiments, the heterologous polynucleotide comprises a hairpin loop sequence comprising a plurality of MS2 hairpin loops and the detectable molecule comprises an MS2 coat binding protein (MCP).

In some embodiments, the heterologous polynucleotide comprises a hairpin loop sequence comprising a PP7 hairpin sequence and the detectable molecule comprises a PP7 coat binding protein.

In some embodiments, the binding motif comprises a hairpin loop sequence or an aptamer sequence and the detectable molecule comprises a U1A RNA-binding protein.

In some embodiments, the binding motif comprises an RNA aptamer sequence and the detectable molecule is a fluorogen. In particular embodiments, the RNA aptamer is a Spinach aptamer or a variant or derivative thereof.

In some embodiments, the promoter is an inducible promoter.

In some embodiments, the cell is a eukaryotic cell (e.g., a mammalian cell (e.g., a human cell)).

In some embodiments, the present disclosure provides a cell comprising an RNA sequence that is prone to forming clusters of RNA, which comprises sequences that form Watson-Crick base pairing (e.g., adenine (A)-thymine (T) or guanine (G)-cytosine (C) interactions), non-canonical base pairing (e.g., interaction between G with U within a secondary structure of RNA) and/or helical stacking (e.g., parallel or antiparallel A-D/B-C RNA helical stacks; parallel or antiparallel A-B/C-D RNA helical stacks).

In some embodiments, the present disclosure provides a cell comprising a heterologous polynucleotide comprising a promoter operably linked to a polynucleotide for encoding an RNA transcript comprising (i) an RNA sequence comprising a sequence that is prone to forming clusters of RNA, which comprises sequences that form Watson-Crick base pairing (e.g., adenine (A)-thymine (T) or guanine (G)-cytosine (C) interactions), non-canonical base pairing (e.g., interaction between G with U within a secondary structure of RNA) and/or helical stacking (e.g., parallel or antiparallel A-D/B-C RNA helical stacks; parallel or antiparallel A-B/C-D RNA helical stacks), and (ii) a binding motif for binding to a detectable molecule; and a heterologous detectable molecule that binds to the binding motif.

In some embodiments, the present disclosure provides a cell comprising an RNA sequence that is prone to forming clusters of RNA, which comprises long non-coding RNAs (lncRNAs), long mRNAs, an RNA transcript of a cluster of microRNAs (pri-miRNA), centromeric transcripts, or RNA transcripts, overexpression and aggregation of which are associated with a disease or disorder, such as nucleotide repeat sequences that are associated with repeat expansion disorders (e.g., CUG repeats in myotonic dystrophy 1).

In some embodiments, the present disclosure provides a cell comprising a heterologous polynucleotide comprising a promoter operably linked to a polynucleotide for encoding an RNA transcript comprising (i) an RNA sequence comprising a sequence that is prone to forming clusters of RNA, which comprises long non-coding RNAs (lncRNAs), long mRNAs, an RNA transcript of a cluster of microRNAs (pri-miRNA), centromeric transcripts, or RNA transcripts, overexpression and aggregation of which are associated with a disease or disorder (such as nucleotide repeat sequences that are associated with repeat expansion disorders, e.g., CUG repeats in myotonic dystrophy 1), and (ii) a binding motif for binding to a detectable molecule; and a heterologous detectable molecule that binds to the binding motif.

In some embodiments, the present disclosure provides a cell comprising an RNA, which is prone to forming clusters, and forms such clusters by aggregating a protein (e.g., a Muscleblind RNA-binding protein, or p53 aggregation modulated by RNAs).

In some embodiments, the present disclosure provides a cell comprising a heterologous polynucleotide comprising a promoter operably linked to a polynucleotide for encoding an RNA transcript comprising (i) an RNA sequence comprising a sequence that is prone to forming clusters of RNA, and forms such clusters by aggregating a protein (e.g., a Muscleblind RNA-binding protein, or p53 aggregation modulated by RNAs), and (ii) a binding motif for binding to a detectable molecule; and a heterologous detectable molecule that binds to the binding motif

In another aspect, the disclosure provides a method of detecting the formation of cellular clusters of RNA. In some embodiments, the method comprises: (a) inducing transcription of the RNA sequence in a cell as disclosed herein, thereby forming transcribed RNAs comprising a sequence that is prone to forming clusters of RNA; and (b) detecting in the cell the formation of one or more clusters of the transcribed RNAs. In some embodiments, the clusters of RNA are mediated by base pairing.

In some embodiments, the detecting step (b) comprises quantifying the amount of clusters of RNA formed in the cell.

In some embodiments, the detecting step (b) comprises detecting the formation of one or more clusters of RNA in the nucleus of the cell.

In another aspect, the disclosure provides a method of identifying an agent that dissolves or inhibits the formation of cellular clusters of RNA. In some embodiments, the method comprises: (a) contacting an agent to a cell as disclosed herein, wherein the cell comprises a plurality of RNA transcripts comprising a sequence that is prone to forming clusters of RNA; (b) quantifying the amount of clusters of RNA formed by the RNA transcripts in the cell that has been contacted with the agent; and (c) comparing the amount of clusters of RNA formed in (b) with a control value, wherein an amount of clusters of RNA formed in (b) that is less than the control value identifies the agent as an agent that dissolves or inhibits the formation of clusters of RNA. In some embodiments, the clusters of RNA are mediated by base pairing.

In some embodiments of this aspect, the control value is an amount of clusters of RNA formed by the RNA transcripts in the cell prior to the contacting step (b).

In some embodiments, the method comprises quantifying the amount of clusters of RNA formed in the nucleus of the cell.

In some embodiments, the agent is a small molecule, an oligonucleotide, or a protein. In particular embodiments, the agent is a nucleic acid intercalator.

In some embodiments, the method further comprises chemically synthesizing a structurally related agent derived from the identified agent.

In another aspect, the disclosure provides structurally related agents of the agents disclosed herein (e.g., an agent identified according to a method disclosed herein).

In yet another aspect, the disclosure provides a method of treating a subject having a disease characterized by clusters of RNA. In some embodiments, the method comprises:

administering to the subject an agent that inhibits or dissolves the formation of clusters of RNA transcripts comprising a sequence that is prone to forming clusters of RNA; thereby treating the subject. In some embodiments, the formation of clusters of RNA in the subject is mediated by base pairing.

In some embodiments, the disease is caused by repeat expansions. In some embodiments, the disease is Huntington's disease, Huntington disease-like 2 (HDL2), myotonic dystrophy, spinocerebellar ataxia, spinal and bulbar muscular atrophy (SBMA), dentatorubral-pallidoluysian atrophy (DRPLA), amyotrophic lateral sclerosis, frontotemporal dementia, Fragile X syndrome, fragile X mental retardation 1 (FMR1), fragile X mental retardation 2 (FMR2), Friedreich's ataxia (FRDA), fragile X-associated tremor/ataxia syndrome (FXTAS), myoclonic epilepsy, oculopharyngeal muscular dystrophy (OPMD), or syndromic or non-syndromic X-linked mental retardation.

In some embodiments, the agent is a small molecule, an oligonucleotide, a protein, or a combination thereof. In particular embodiments, the agent is an intercalating agent.

In some embodiments, the method comprises administering to the subject a pharmaceutical composition comprising a small molecule, an oligonucleotide, a protein, or a combination thereof.

### BRIEF DESCRIPTION OF THE DRAWINGS

FIG. 1A shows a schematic illustrating the intramolecular hairpins formed from G and C nucleotide base pairing in an RNA.

FIG. 1B shows representative fluorescence micrographs of micrometre-sized spherical clusters formed in RNAs having 47 repeats of CUG or 47 repeats of CAG and quantification of inhomogeneity as normalized variance (σ²/μ).

FIG. 1C shows representative fluorescence micrographs for RNA with various GC content compared against RNA with disease-associated repeat expansions. Scale bars: 5 μm.

FIG. 1D shows representative fluorescence micrographs comparing 47× CUG RNA and a corresponding control RNA (Scr1) with identical base composition but with a scrambled sequence. Similarly, 47× CAG RNA was compared with two different RNAs (Scr2, Scr3) having the same base composition as 47× CAG but whose sequences were scrambled. The extent of inhomogeneity was quantified by the index of dispersion (σ²/μ) across more than 20 independent imaging areas (1,800 μm² each). Each data point represents an independent imaging area. Scale bars: 5 μm.

FIG. 1E shows representative fluorescence micrographs of 47× CAG RNA clusters at indicated concentrations. Spherical RNA clusters are observable up to 25 nM RNA concentration. In this concentration regime, the reaction is reactant limited and the cluster size is below the diffraction limit. RNA clustering at all concentrations was not observed in the presence of 100 mM ammonium acetate. Representative images at the indicated RNA concentration in the presence of 100 mM NH₄OAc (+NH₄OAc). Scale bars: 5 μm.

FIG. 1F shows RNA enrichment in the clusters. Left, Cy3-labelled 66× CAG RNA was serially diluted in conditions preventing RNA clustering (10 mM Tris pH 7.0, 10 mM MgCl₂, 100 mM NaCl), and the bulk solution fluorescence was calibrated against the RNA concentration. The enrichment of RNA in the clusters was determined by comparing their fluorescence intensity against this calibration. When the input RNA concentration was 100 ng per μL, the concentration in the clusters corresponded to about 16.3 μg/μL, or an enrichment of 163-fold (a.u., arbitrary units). Right, RNA clusters were precipitated by centrifugation at 16,000 g for 10 minutes at room temperature. The concentration of the soluble RNA after centrifugation was determined by measuring absorbance at 260 nm. The concentration of the RNA in the solution phase decreased with the increasing CAG-repeat number.

FIGS. 1G and 1H show representative fluorescence micrographs and quantification of 47× CAG RNA clusters, which were treated with proteinase K (60 U/mL), DNase I (200 U/mL), or RNaseA (0.7 U/mL) for 10 minutes at room temperature. Scale bars: 5 μm.

FIG. 11 shows representative fluorescence micrographs and quantification of clustering of 47× CAG RNA that was inhibited by NaCl. Scale bars: 5 μm.

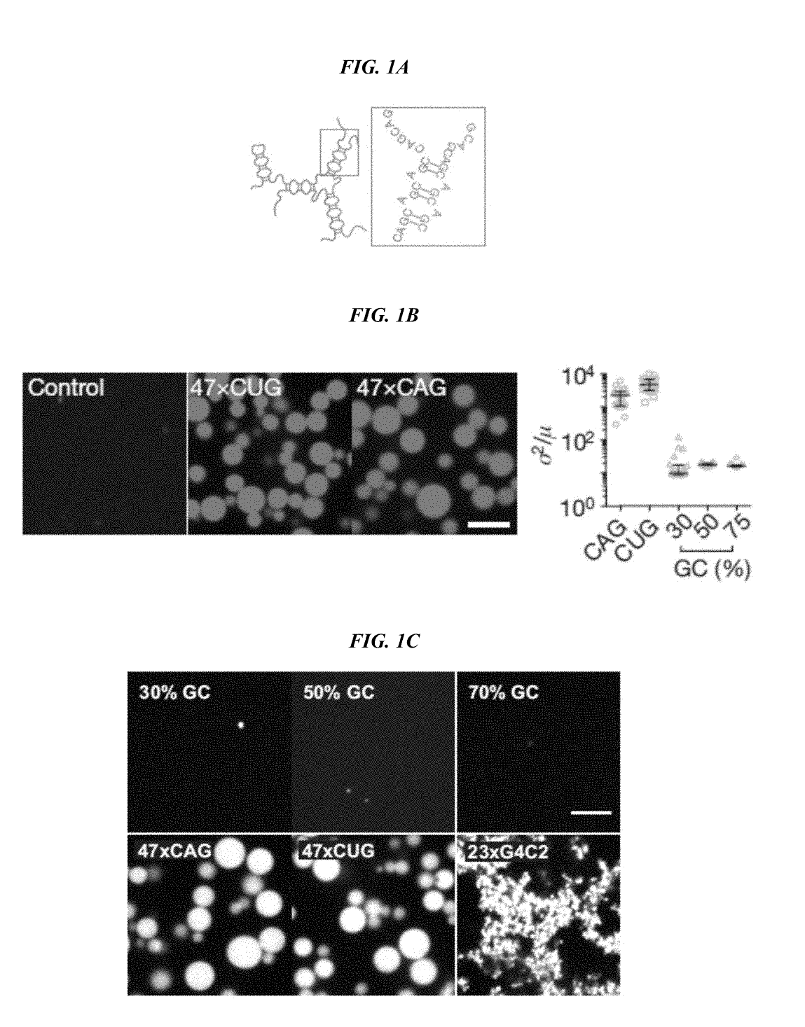

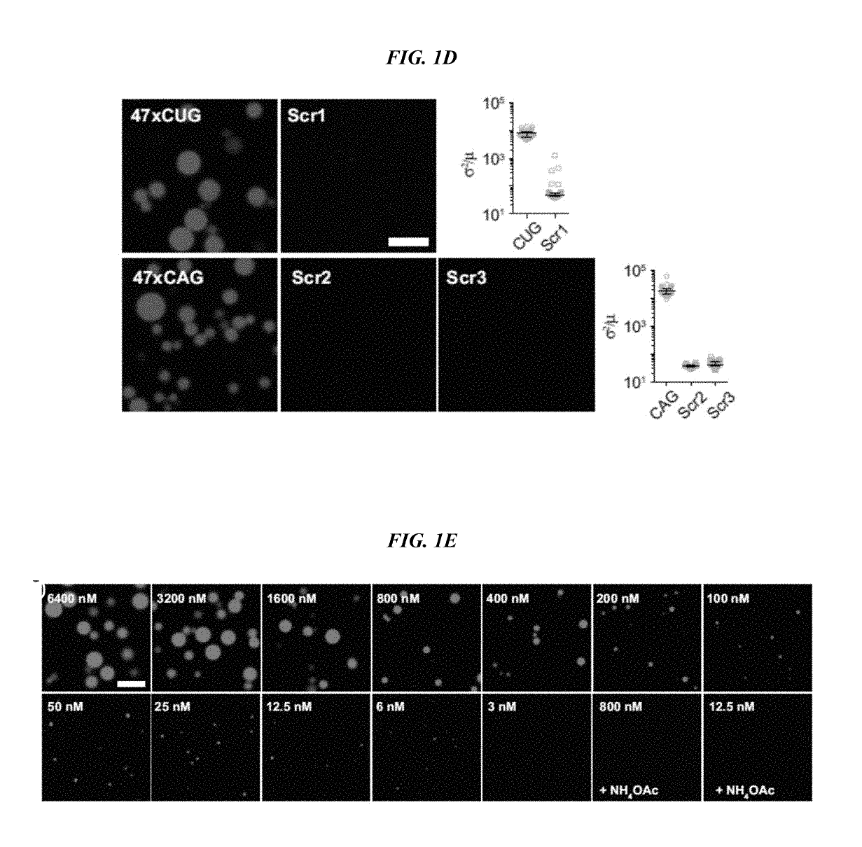

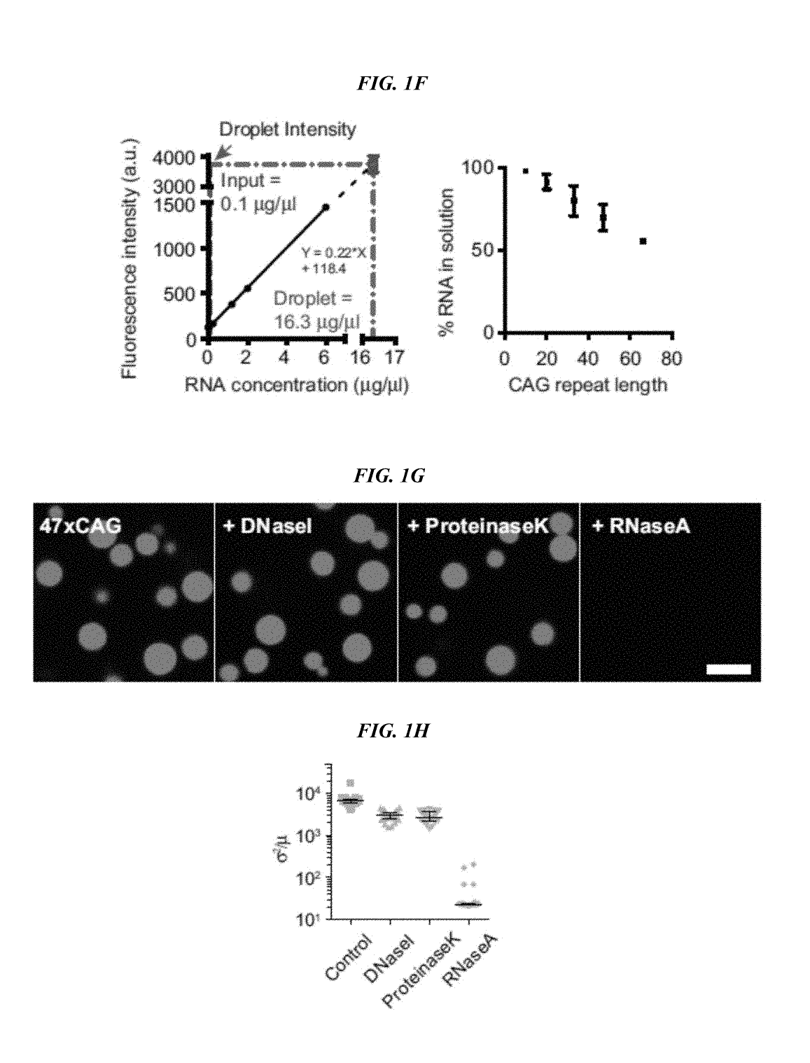

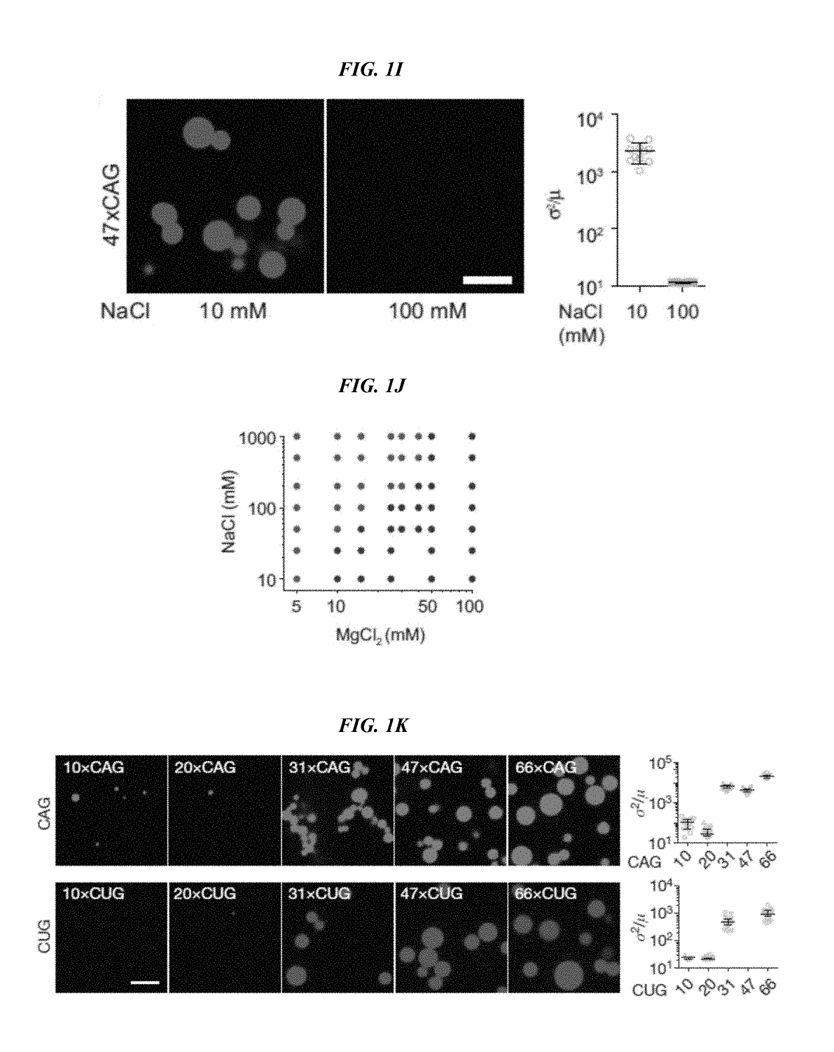

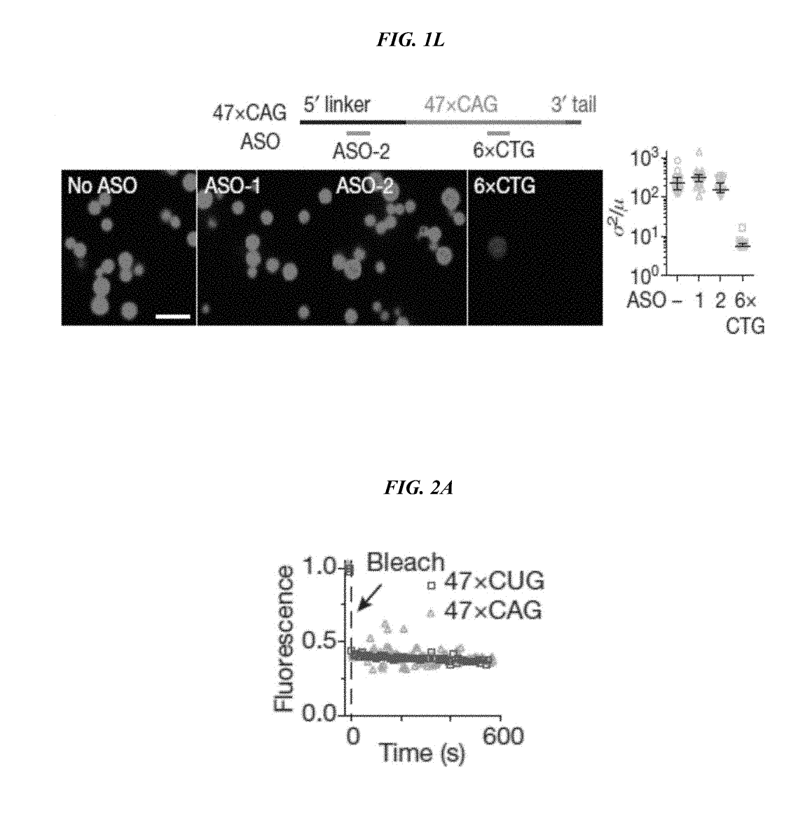

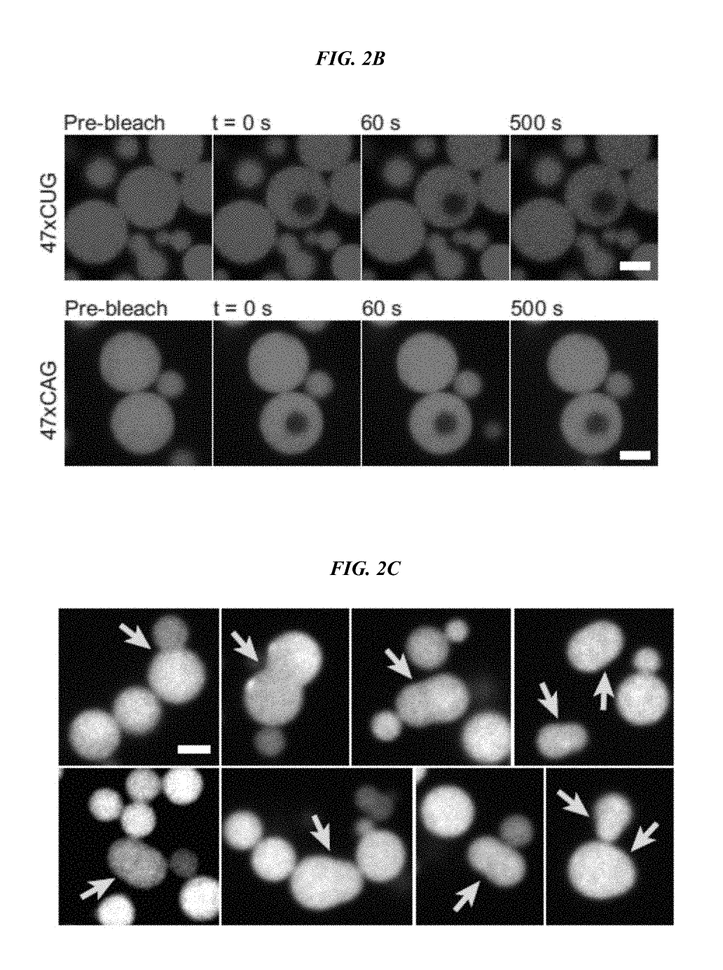

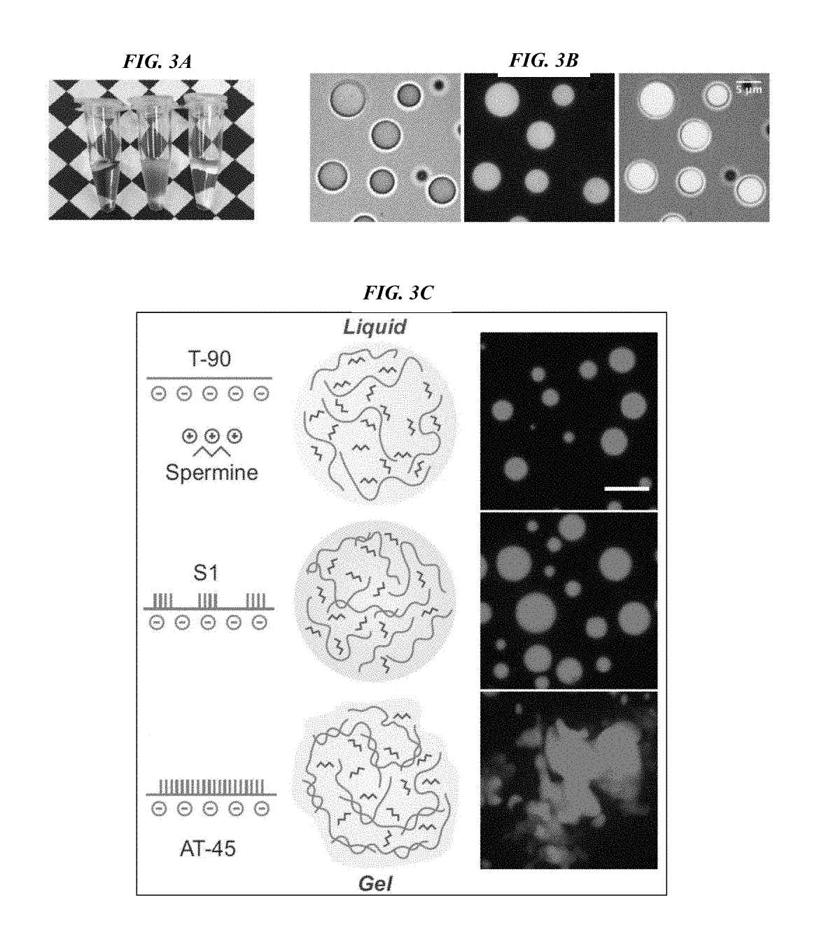

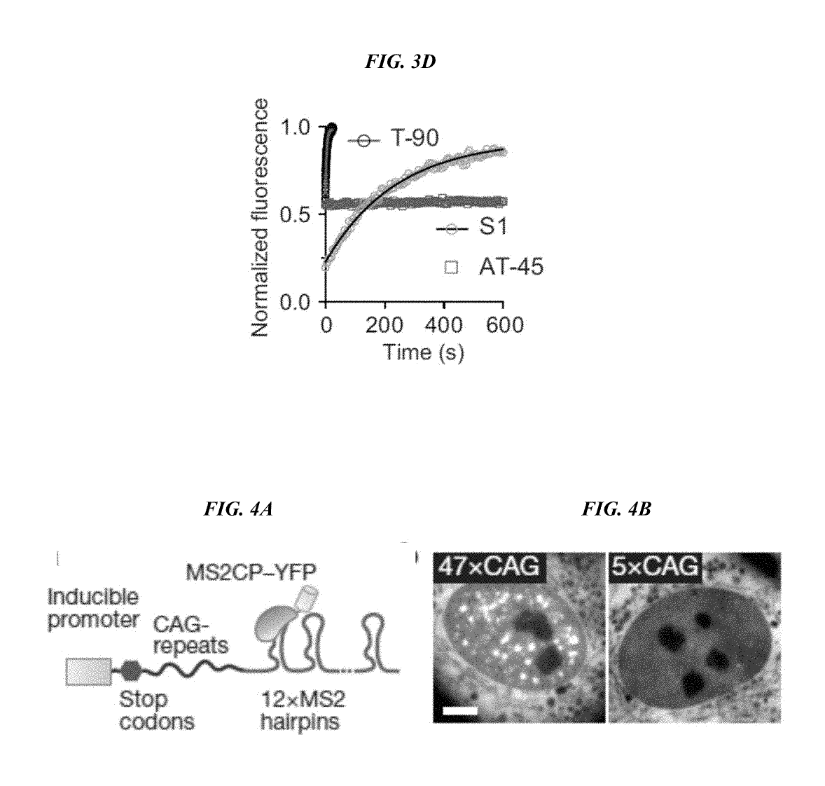

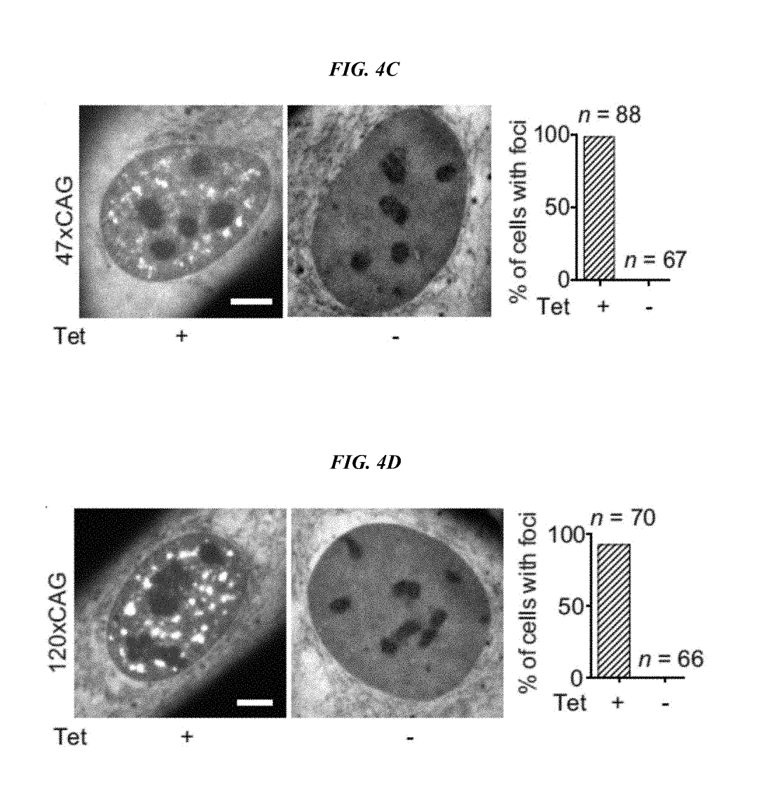

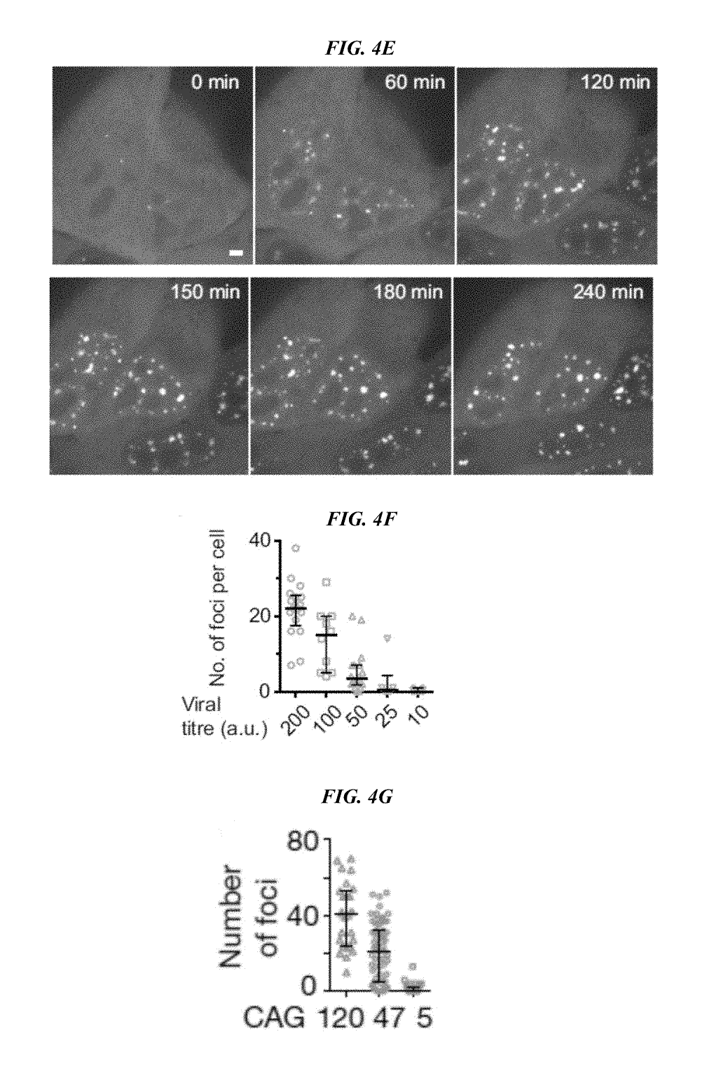

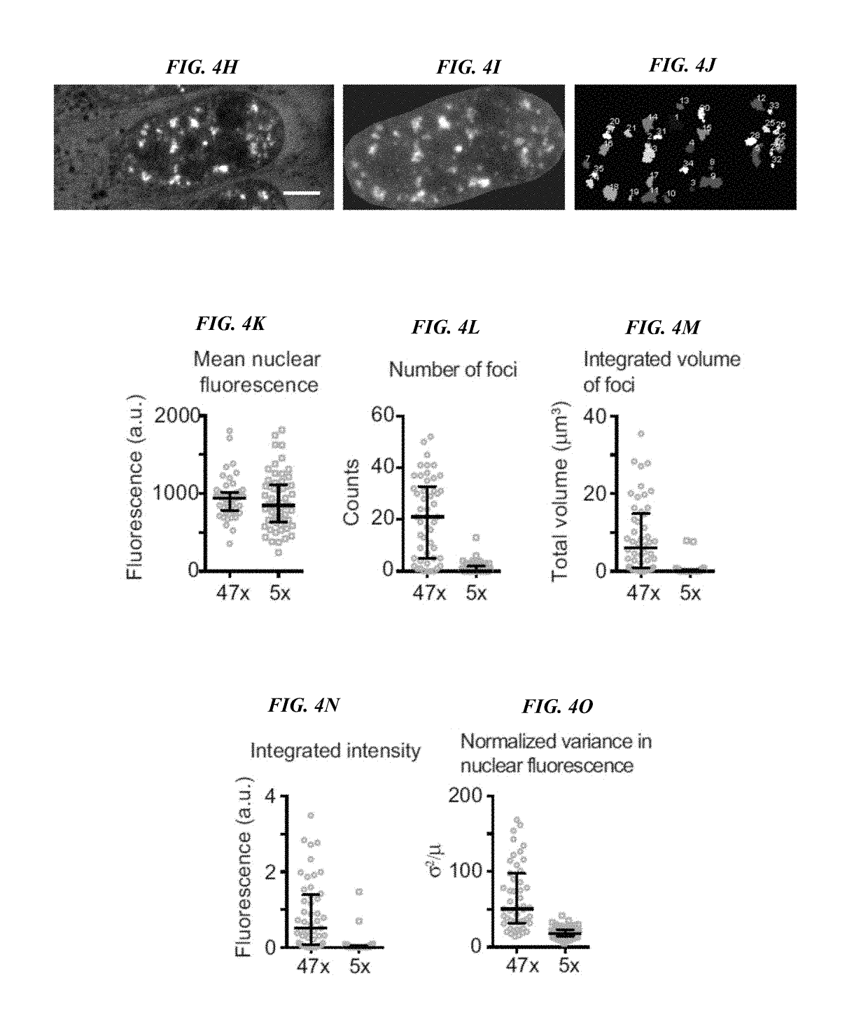

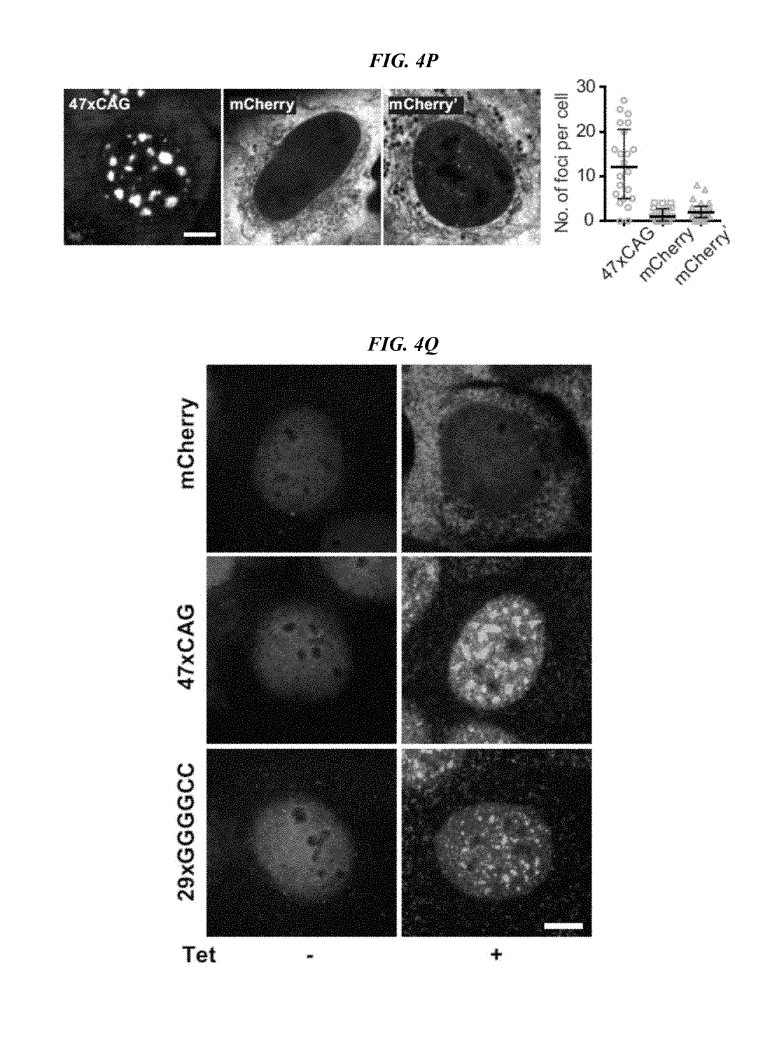

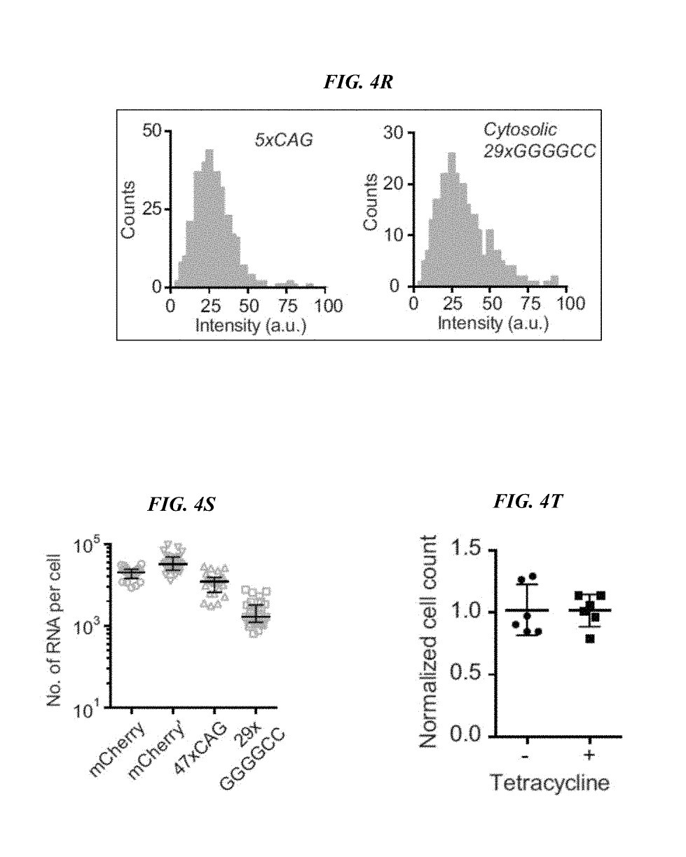

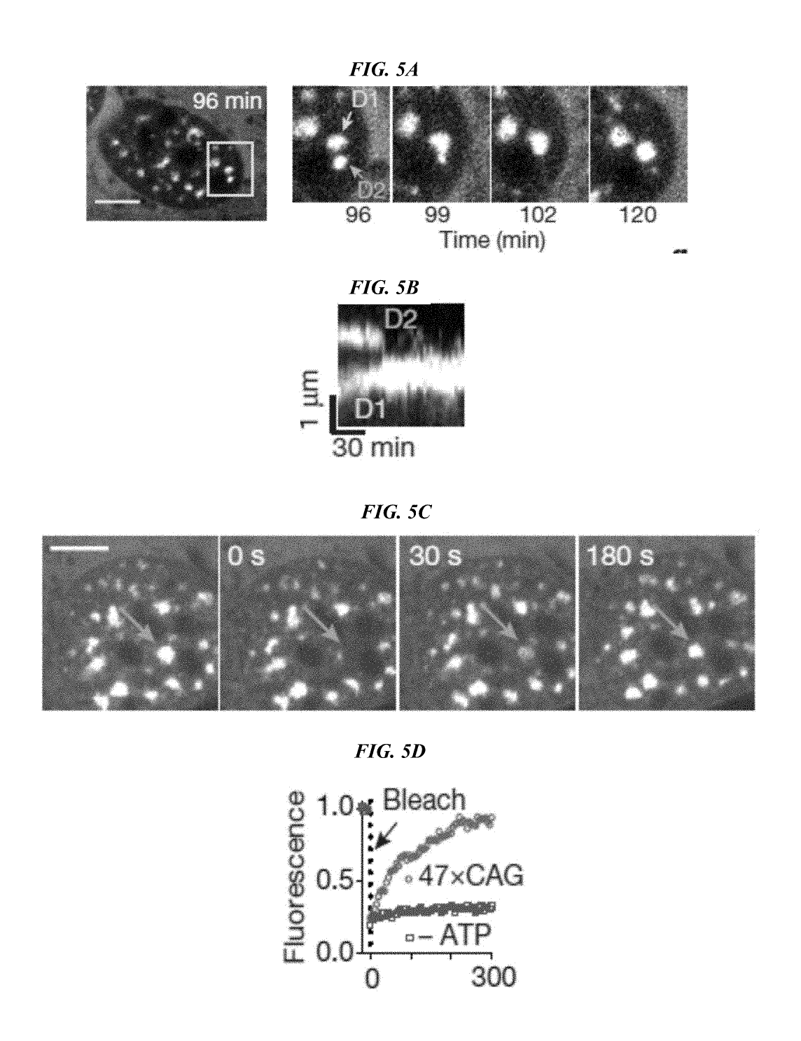

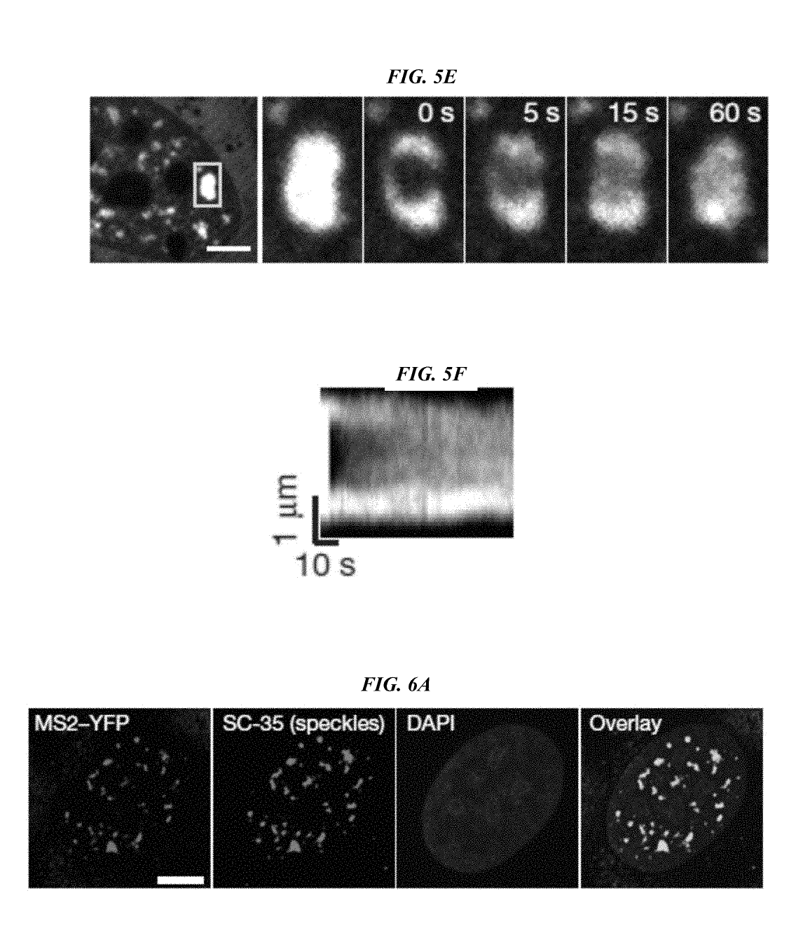

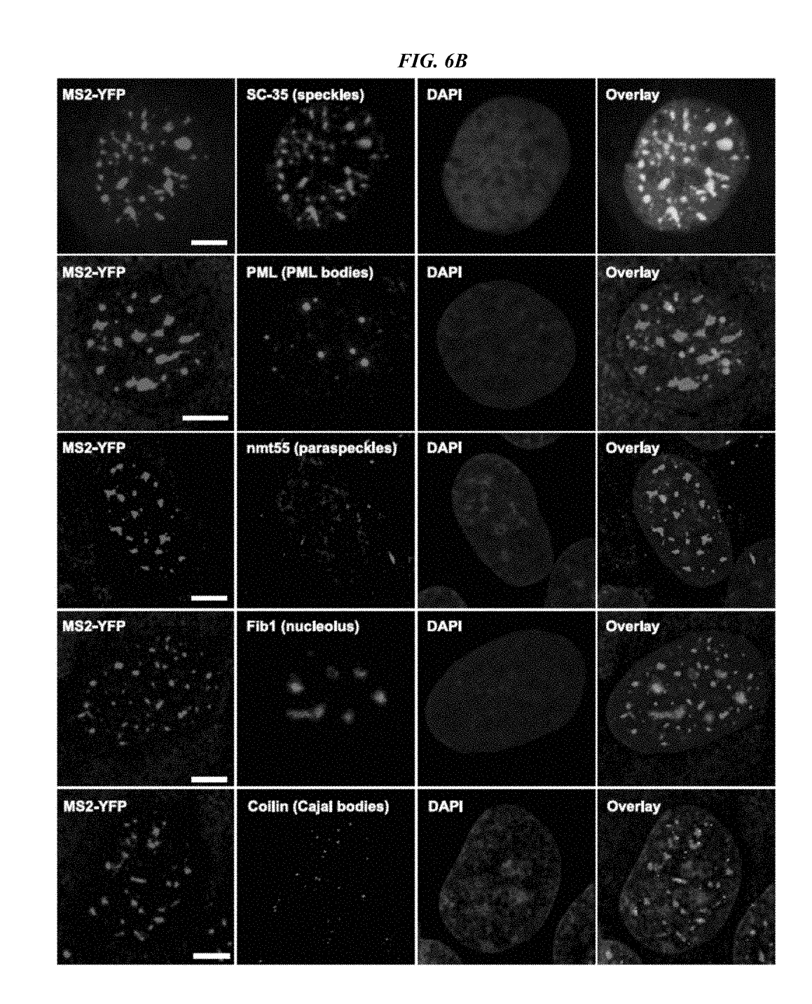

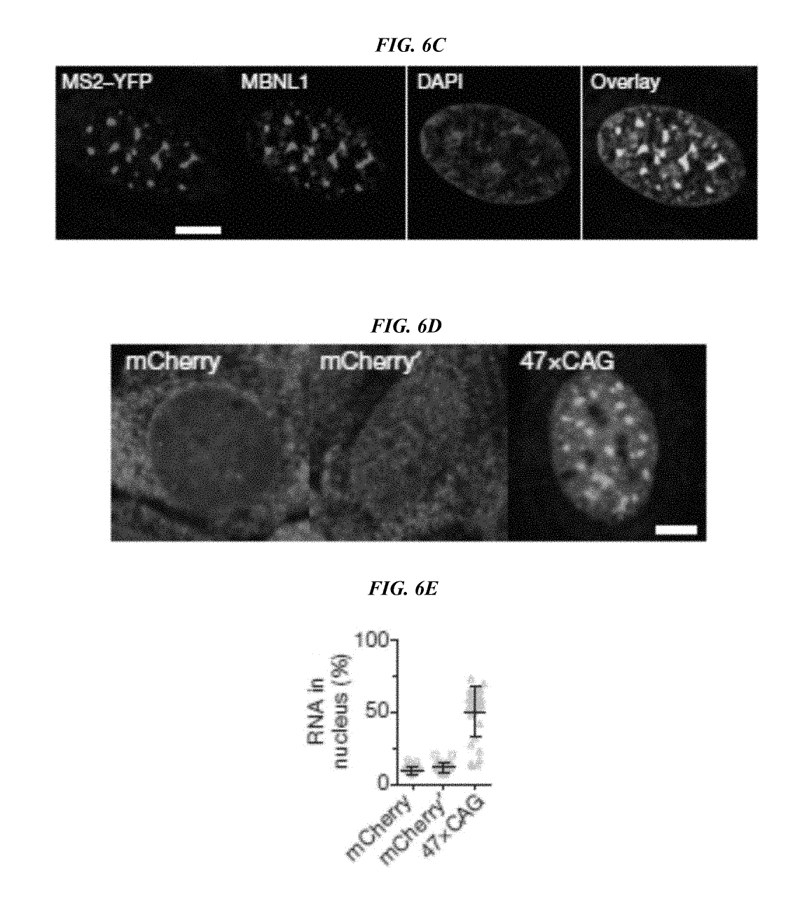

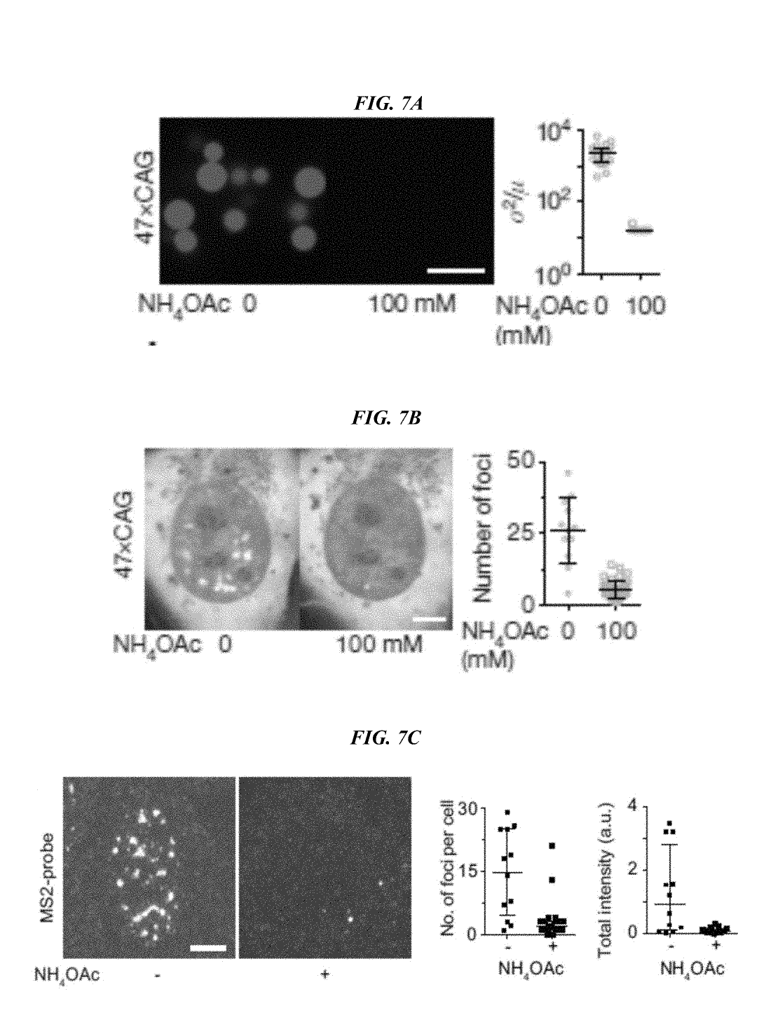

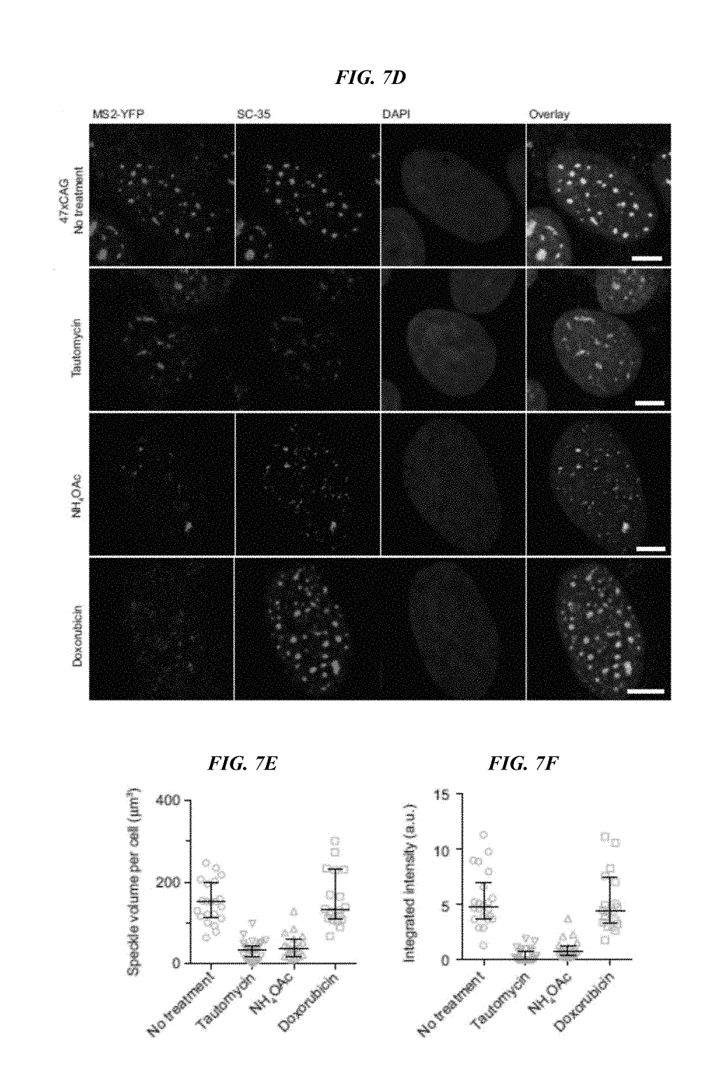

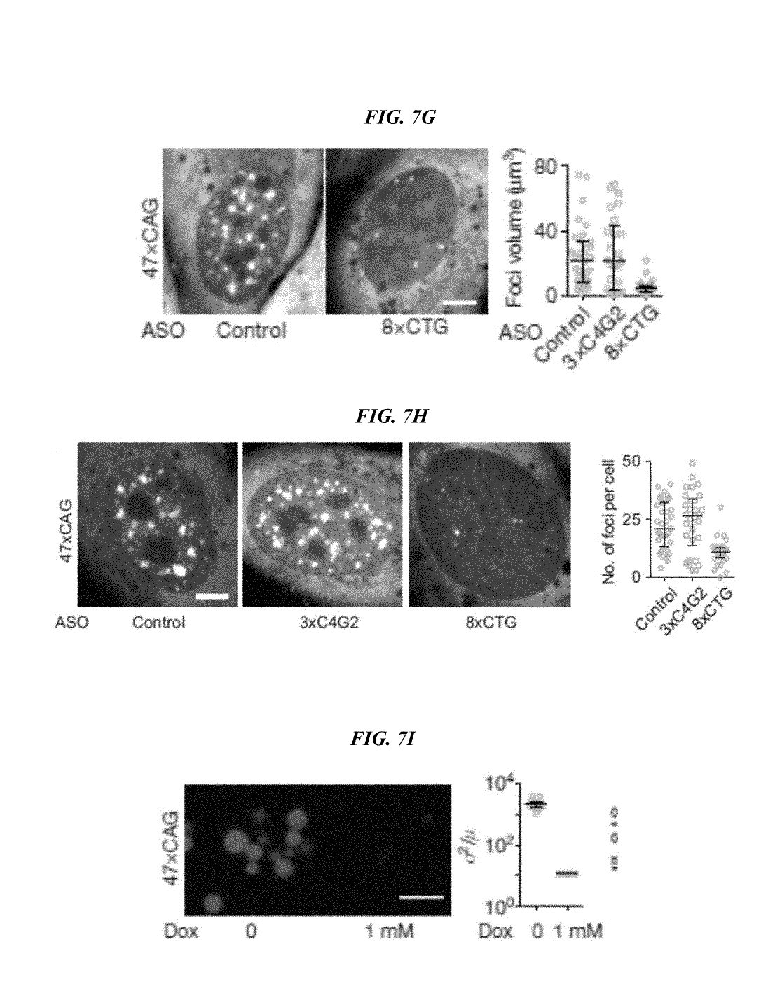

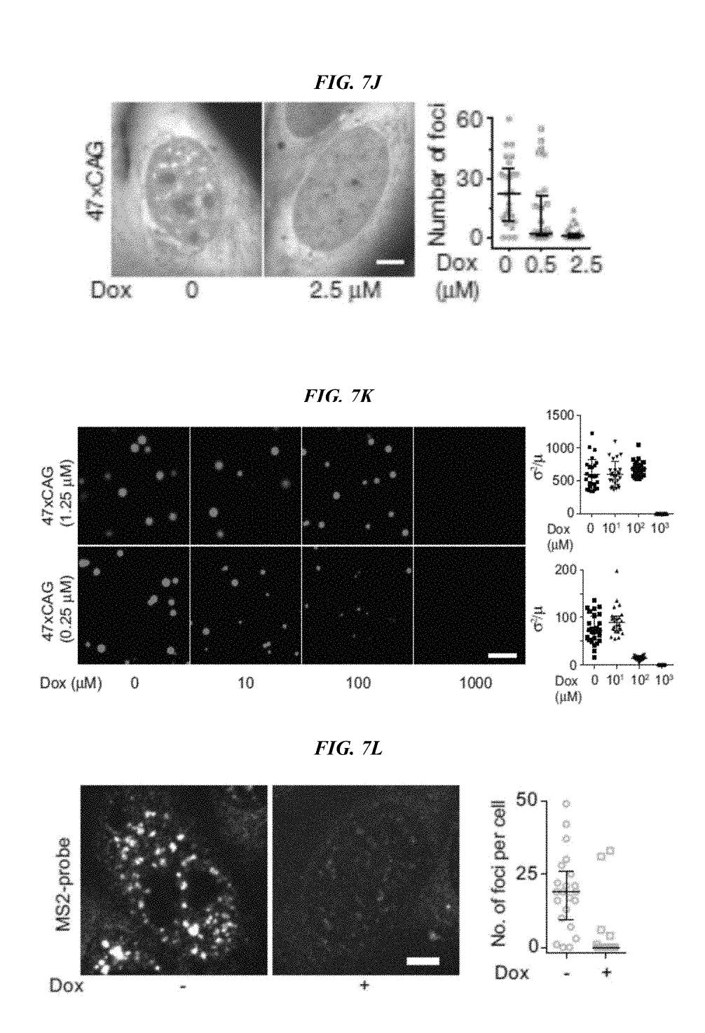

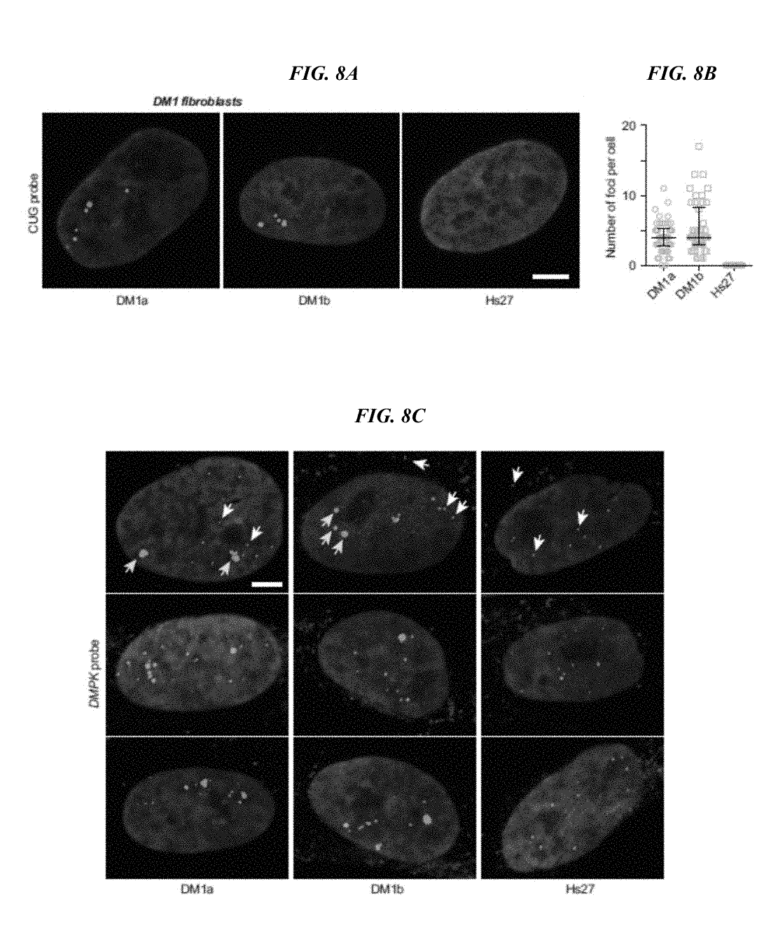

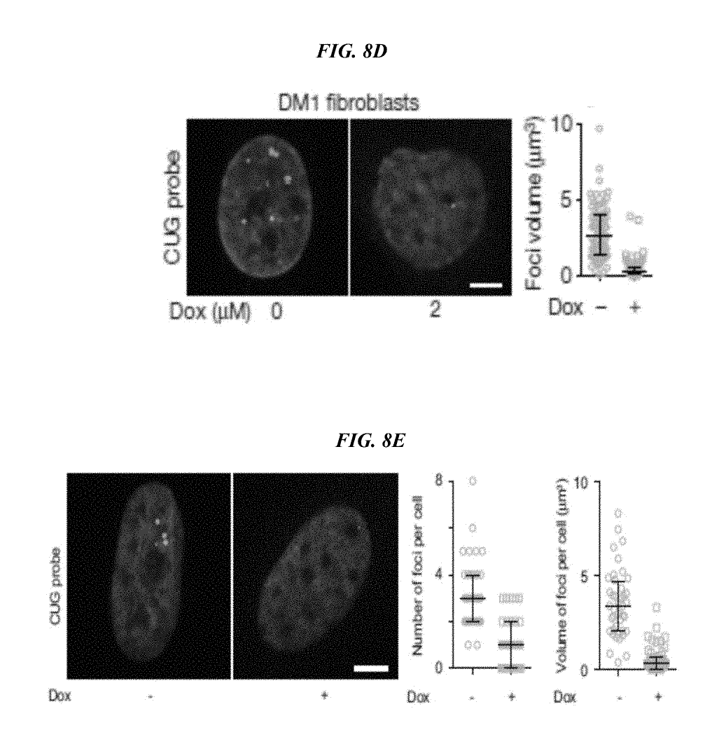

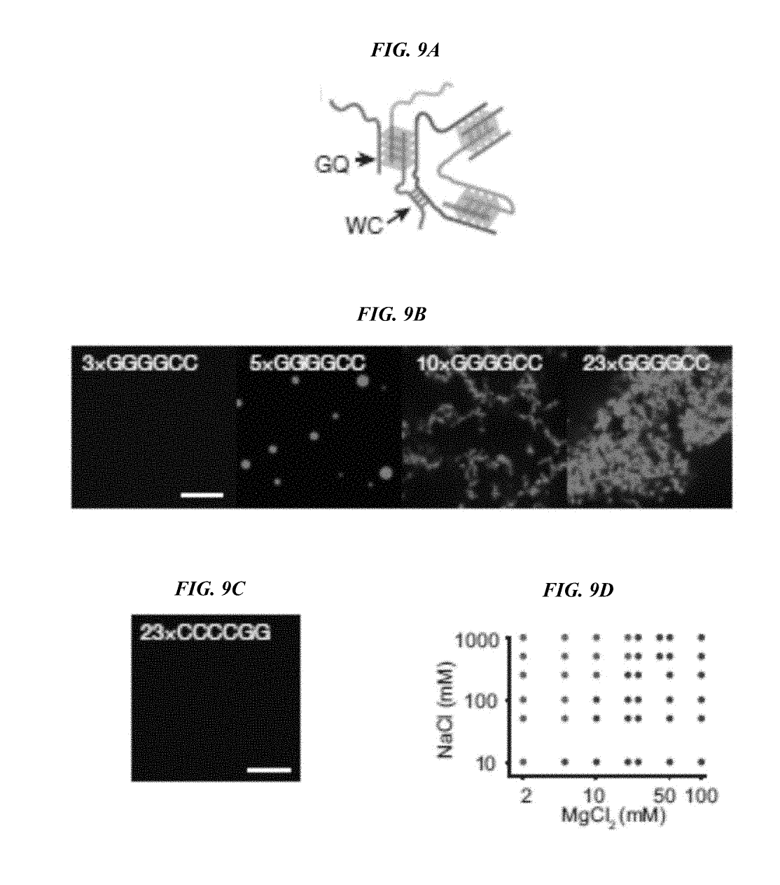

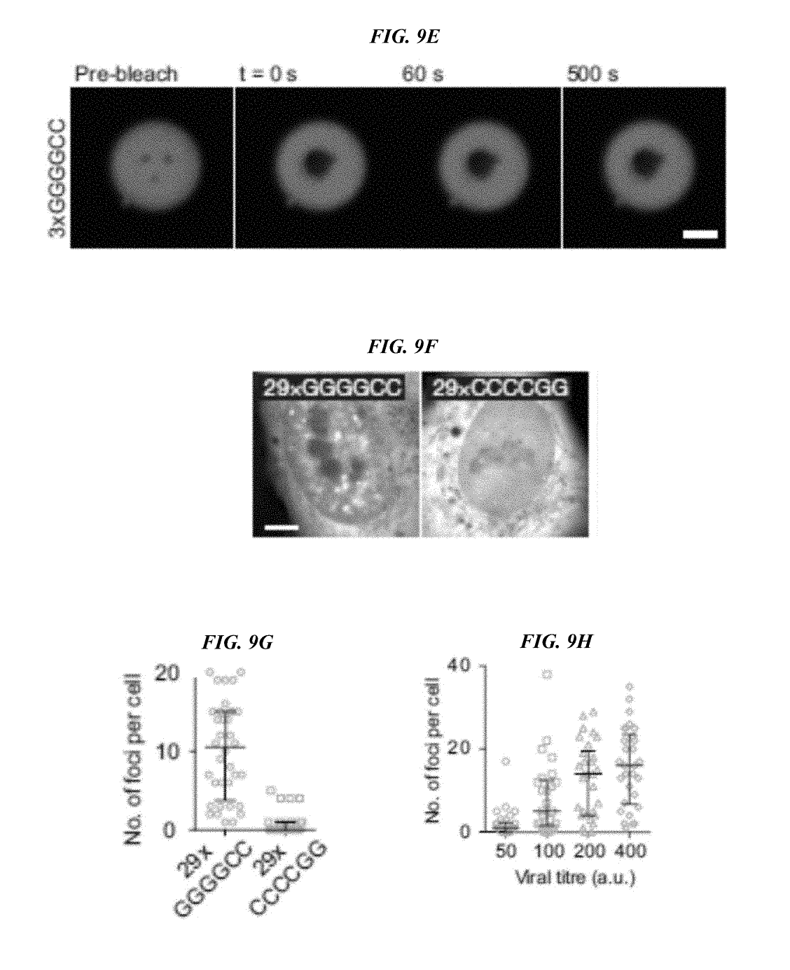

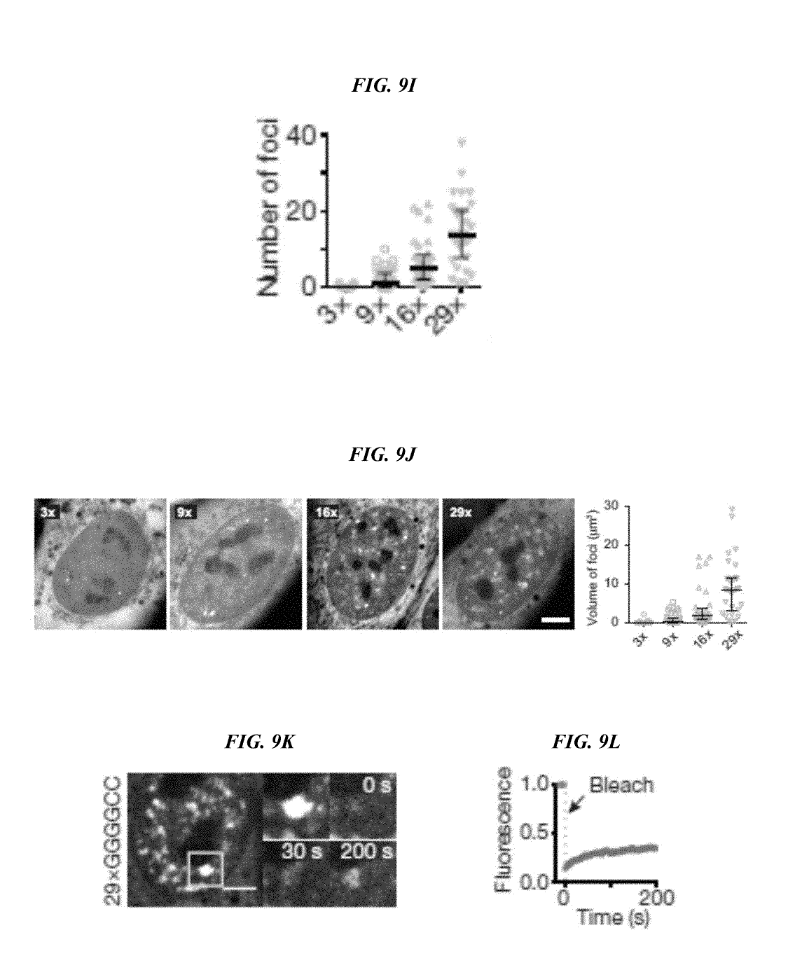

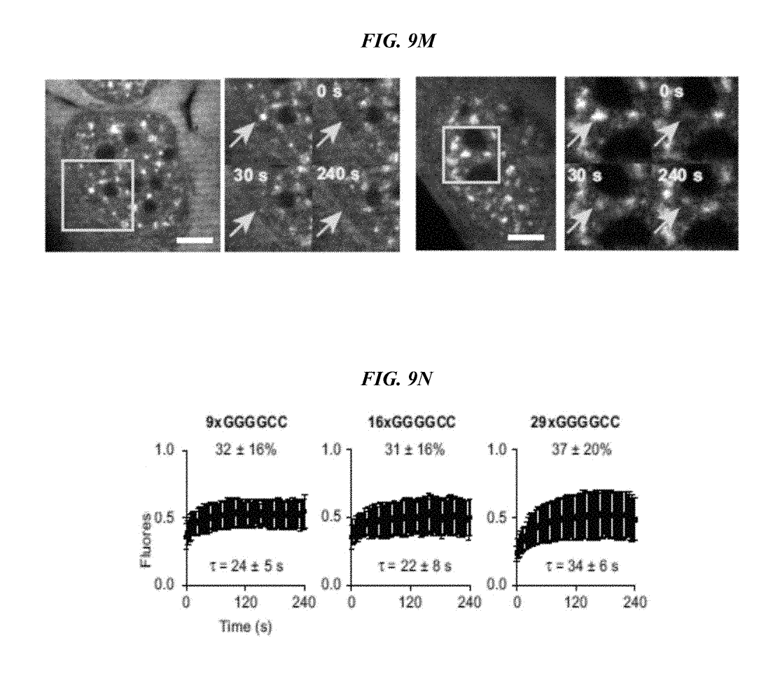

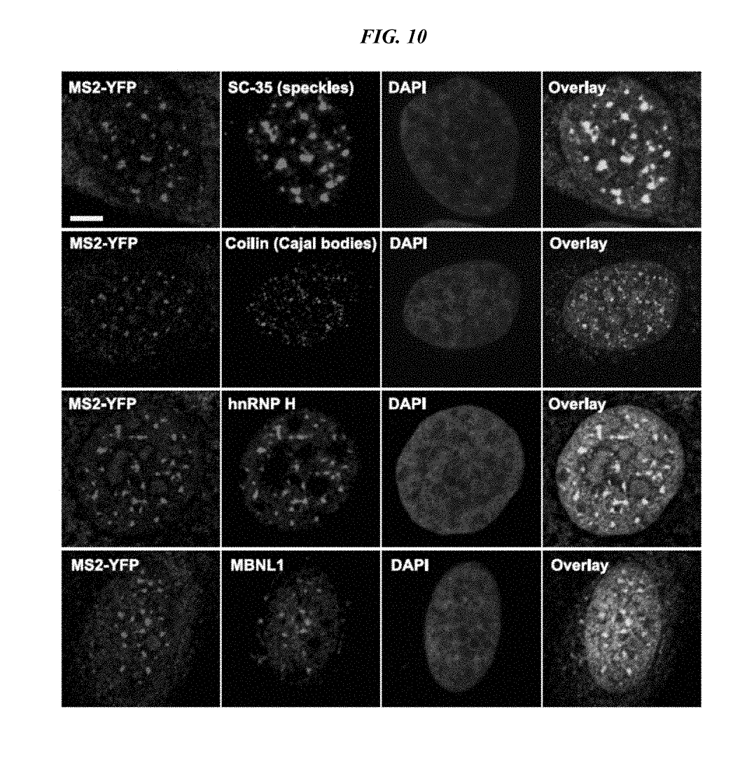

FIG. 1J shows binary phase diagram for 1.25 μM 47× CAG RNA as a function of MgCl₂ and NaCl concentrations. Blue dots represent two-phase regime while the red dots indicate a homogenous single-phase regime.

FIG. 1K shows representative fluorescence micrographs and quantification of the effects of CAG- or CUG-repeat number on RNA gelation.

FIG. 1L shows representative fluorescence micrographs and quantification of the effects of control ASO (ASO-2, and non-hybridizing oligonucleotide, ASO-1) or 6× CTG on 47× CAG on RNA clustering.

FIG. 2A shows FRAP trajectories for RNA clusters. Scale bars, 5 μm. Data are median±interquartile range. Data are representative of at least three independent experiments.

FIG. 2B shows that the RNA clusters were in a solid-like state and did not exhibit fluorescence recovery upon photobleaching, as indicated by the representative fluorescence micrographs for 47× CUG (top) and 47× CAG (bottom) RNA at the indicated time points. Scale bars: 1 μm.

FIG. 2C shows representative images showing aborted fusion events between 47× CAG RNA clusters, suggesting that the clusters were liquid-like initially and later underwent a liquid-to-solid transition. Fusion events were probably aborted as the clusters solidified before relaxation to a spherical geometry. Sites of aborted fusion are marked by arrows. Scale bars: 1 μm.

FIG. 3A shows that electrostatic interactions between polymeric anions (such as nucleic acids) and multivalent cations can lead to phase separation via formation of polyelectrolyte complexes, a phenomenon known as complex coacervation. It was found that spermine, a tetravalent cation at pH 7, could induce phase separation of single-stranded DNA oligonucleotides. Mixing 10 mM spermine pH 7 (left tube) with 10 μM T-90 DNA (90-mer polyT DNA oligonucleotide) (right tube) immediately resulted in a turbid solution (center tube).

FIG. 3B shows representative bright-field (left), fluorescence (center), and overlay (right) images for the DNA-spermine complexes. Examination by bright-field microscopy revealed numerous spherical droplets. Using a fluorescently labelled T-90, it was confirmed that the droplet phase was enriched in DNA.

FIG. 3C shows that the T-90 DNA-spermine complexes were liquid-like, as evidenced by their spherical geometry (top panel). A 90-base-long DNA with five 8-bp palindromic hybridization sites separated by poly-dT spacers (sequence 51) also phase-separated and formed spherical liquid-like droplets in the presence of spermine (middle panel). A (dAdT)45 oligonucleotide (AT-45) that could form multivalent A:T base-pairing interactions was also tested in a similar spermine-mediated coacervation experiment. AT-45 DNA formed interconnected network-like structures spanning hundreds of micrometers or gels (bottom panel). Scale bars, 5 μm.

FIG. 3D shows that T-90 DNA-spermine complexes displayed a rapid FRAP (99±1% recovery, τ_{FRAP}−T90=5±2 s, mean±s.d., n=3 droplets). S1-spermine droplets exhibited reduced fluidity, as evidenced by a slower recovery upon photobleaching (90±4% recovery, τ_{FRAP-S1}=335±41 s, mean±s.d., n=5). The AT-45 DNA gels were in a solid-like state, as evidenced by lack of FRAP (14±5% recovery, mean±s.d., n=4 clusters).

FIG. 4A shows a schematic illustrating RNA visualization experiment.

FIG. 4B shows representative micrographs of cells expressing 5× CAG or 47× CAG RNA.

FIGS. 4C and 4D show representative micrographs of cells expressing 47× CAG (FIG. 4C) or 120× CAG (FIG. 4D).

FIG. 4E shows time-lapse images of 120× CAG RNA accumulation in the nuclei of U-2OS cells. Cells were induced with 1 μg/mL of doxycycline at t=0.

FIG. 4F shows that the number of foci per cell increased with increasing 47× CAG RNA expression levels. The expression levels were controlled by increasing the virus titer. Each data point represents one cell and the data are shown as median±interquartile range.

FIG. 4G shows the quantification of foci as a function of CAG-repeat number. Each data point represents one cell. Data are median±interquartile range.

FIGS. 4H-4J show identification of RNA foci in live cells. A fluorescence-intensity and size-based threshold was used to identify RNA foci. In brief, U-2OS cells expressing the RNA of interest together with MS2CP-YFP were imaged using a spinning disk confocal microscope, and 0.3 μm Z-stacks were acquired (FIG. 4H). To account for variability in MS2CP-YFP expression levels, a cell-intrinsic intensity threshold was used for foci identification. The nuclei were manually segmented (FIG. 41) and the mean fluorescence intensity in the nucleus was determined. RNA foci were identified using the FIJI 3D Objects Counter plugin, with an intensity threshold as 1.6× the mean fluorescence intensity in the nucleus of the cell, and a size cut-off of more than 50 adjoining pixels (pixel size, 83 nm×83 nm). This algorithm accurately identified the foci as depicted in FIG. 4J. Scale bar, 5 μm.

FIGS. 4K-4O show the comparison of the extent of foci formation in 47× CAG and 5× CAG expressing cells. The mean nuclear fluorescence intensity was similar between the 47× CAG and 5× CAG expressing cells (FIG. 4K). The cells were compared via various metrics: number of foci per cell (FIG. 4L); total volume of foci per cell (FIG. 4M); integrated fluorescence intensity of the foci per cell (FIG. 4N); and normalized variance in the fluorescence intensity in the nucleus per cell (FIG. 4O). Data are median±interquartile range.

FIG. 4P shows that 47× CAG RNA accumulated in the nuclei as puncta, while control RNA with coding (mCherry) or a non-coding sequence (mCherry', reverse complement of mCherry sequence) did not form nuclear inclusions, as shown in the representative MS2-YFP fluorescence micrographs and quantification of the number of foci per cell. Each data point represents one cell and the data are shown as median±interquartile range.

FIG. 4Q shows representative micrographs showing the localization of mCherry (top panels), 47× CAG (middle panels), and 29× GGGGCC (bottom panels) RNA with (+Tet) or without (−Tet) doxycycline induction. The probes did not bind in the absence of induction. U-2OS cells were transduced with the indicated constructs tagged with 12× MS2 hairpins under a tetracycline-inducible promoter. RNA was visualized by FISH using Cy3-labelled oligonucleotide probes against MS2-hairpins. Nuclei are counterstained with DAPI (depicted in blue). Scale bars, 5 μm.

FIG. 4R shows intensity distribution for single RNA spots in cells expressing 5× CAG (left) and in the cytoplasm of cells expressing 29× GGGGCC (right).

FIG. 4S shows the quantification of the micrographs of FIG. 4Q. RNA copy number was determined by dividing the total Cy3 fluorescence intensity in a cell by that of a single RNA, as determined in FIG. 4R. The 47× CAG RNA copy number corresponds to the highest viral titre used in FIG. 4F. Similar results were obtained using NanoString (8,800±1,500 copies per cell for 47× CAG RNA, mean±s.d., n=3 independent experiments). Each data point represents one cell and the data are shown as median±interquartile range.

FIG. 4T shows normalized cell counts in 47× CAG-transduced cells with or without doxycycline induction. The induction of 47× CAG RNA foci did not cause overt toxicity or a reduction in cell division rates over 7 days. Cell counts were normalized to control cells (without 47× CAG transduction), grown under corresponding induction conditions. Data points represent technical replicates, and are shown as mean±s.d.

FIGS. 5A and 5B show a typical fusion event between RNA foci (time after induction indicated) and the corresponding kymograph, respectively. Scale bars, 5 μm.

FIG. 5C shows representative images for 47× CAG RNA foci before and after photobleaching (arrow, bleach site). Scale bars, 5 μm.

FIG. 5D shows FRAP trajectories for 47× CAG RNA punctum before (47× CAG) and after ATP depletion (−ATP).

FIGS. 5E and 5F show representative images for 47× CAG RNA foci before and after partial photobleaching and the corresponding kymograph, respectively. Scale bars, 5 μm.

FIG. 6A shows representative immunofluorescence images depicting co-localization of 47× CAG RNA foci (MS2-YFP) with nuclear speckles (SC-35). Nuclei are counterstained with 4′,6-diamidino-2-phenylindole (DAPI).

FIG. 6B shows representative immunofluorescence micrographs depicting that the 47× CAG RNA foci co-localized with the marker for nuclear speckles (SC-35) but not with other nuclear bodies such as PML bodies (PML), paraspeckles (nmt55), nucleoli (Fib1), or Cajal bodies (coilin). RNA foci were stained using an antibody against GFP. Nuclei were stained with DAPI. Data are representative of three or more independent experiments. Scale bars, 5 μm.

FIG. 6C shows representative immunofluorescence images depicting co-localization of 47× CAG RNA foci (MS2-YFP) with MBNL1. Nuclei are counterstained with 4′,6-diamidino-2-phenylindole (DAPI).

FIGS. 6D and 6E show fluorescence in situ hybridization (FISH) images and relative RNA abundance per nucleus in cells expressing MS2-tagged 47× CAG, a control coding sequence (mCherry), or its reverse complement (mCherry'). Scale bars, 5 μm. Data are mean ±s.d. Data are representative of at least three independent experiments.

FIG. 7A shows representative fluorescence micrographs showing that NH₄OAc prevents 47× CAG RNA clustering.

FIG. 7B shows representative images of the same cell as shown in FIG. 7A before and 300 s after treatment with NH₄OAc, and corresponding quantification.

FIG. 7C shows representative images and corresponding quantification showing that RNA FISH using a probe directed against MS2 hairpin loops confirmed that 47× CAG RNA foci were disrupted by treatment with 100 mM NH₄OAc, thus precluding the possibility that the observed disruption of RNA foci in live cells was due to dissociation of MS2CP-YFP from the MS2 hairpins.

FIG. 7D shows representative images showing that doxorubicin disrupts RNA foci but not nuclear speckles. First row shows representative immunofluorescence micrographs of U-2OS cells expressing 47× CAG stained with antibodies against GFP (MS2-YFP) and SC-35, as a marker for nuclear speckles. Second and third rows show that treatment with 2 μM tautomycin for 4 hours (second row) or 100 mM NH₄OAc for 10 minutes (third row) disrupted both the 47× CAG RNA foci as well as the nuclear speckles. Fourth row shows that treatment with 2.5 μM doxorubicin for 2 hours specifically abrogated the 47× CAG RNA foci but the nuclear speckles were not disrupted. Nuclei were counterstained with DAPI (blue). Scale bars, 5 μm.

FIGS. 7E and 7F show the quantification of the total volume occupied by nuclear speckles (FIG. 7E) and the integrated intensity of the SC-35 immunofluorescence (FIG. 7F) per cell under various treatments. Data are median±interquartile range. Data are representative of three or more independent experiments.

FIG. 7G shows representative images and quantification of RNA foci upon transfection with ASO.

FIG. 7H shows representative images and quantification of the number of RNA foci per cell. Transfection of an 8× CTG oligonucleotide disrupted 47× CAG RNA foci while control oligonucleotides (3× C4G2 or Control) did not.

FIG. 7I shows representative fluorescence micrographs showing that doxorubicin (Dox) prevents 47× CAG RNA clustering.

FIG. 7J shows representative images of the same cell as shown in FIG. 7I before and 300 s after treatment with DOX, and corresponding quantification.

FIG. 7K shows representative micrographs and the quantification of the inhomogeneity in the solution at indicated RNA and doxorubicin concentrations. Doxorubicin (Dox) disrupted 47× CAG RNA clustering in vitro in a dose-dependent manner.

FIG. 7L shows that RNA FISH using a probe directed against MS2 hairpin loops confirmed that 47× CAG RNA foci were disrupted by treatment with 2.5 μM doxorubicin, suggesting that the observed disruption of RNA foci in live cells was probably not an artifact of MS2CP-YFP dissociation from MS2 hairpins. Scale bars, 5 μm. Data are median±interquartile range. Data are representative of three or more independent experiments.

FIGS. 8A and 8B show representative fluorescence images and quantification showing RNA foci in DM1 cell lines but not in control. Fibroblasts derived from patients with DM1 (DM1a and DM1b) or control fibroblasts (Hs27) were stained using a FISH probe directed against expanded CUG repeats (8× CAG labelled with Atto647N). Nuclei were counterstained with DAPI (blue).

FIG. 8C shows single-molecule FISH using probes designed against the wild-type DMPK allele showed isolated diffraction-limited spots in control fibroblasts (Hs27), indicated by white arrows, probably arising because of single mRNA. In the patient-derived fibroblasts (DM1a and DM1b), isolated spots (white arrows) as well as several bright puncta (yellow arrows) were observed. Since both the wild-type and the mutant transcript with expanded CUG repeats could each accommodate the same number of fluorescent probes (48 probes per transcript), the higher brightness indicates that each punctum in cells derived from patients with DM1 (yellow arrows) contained multiple DMPK mRNAs.

FIG. 8D shows representative images and quantification of RNA foci in fibroblasts from patients with DM1.

FIG. 8E shows representative images with or without doxorubicin treatment and corresponding quantification of RNA foci. Treatment with 2 μM doxorubicin for 24 hours reduced the average number of RNA foci per cell by 66% and the total volume of foci per cell by 87%. Data are aggregated from two independent experiments, and are representative of four or more independent experiments. Scale bars, 5 μm. Data are median±interquartile range.

FIG. 9A shows a schematic illustrating GGGGCC RNA multimerization. GQ, G-quadruplex; WC, Watson-Crick.

FIG. 9B shows representative images of RNA clusters at the indicated number of GGGGCC repeats.

FIG. 9C shows that repeat 23× CCCCGG is soluble.

FIG. 9D shows a binary phase diagram for 23× GGGGCC RNA clustering in vitro as a function of NaCl and MgCl₂ concentrations. RNA concentration was 1.5 μM. Blue dots represent two-phase regime while the red dots indicate a homogenous single-phase regime.

FIG. 9E shows representative fluorescence micrographs for 3× GGGGCC RNA clusters before and after photobleaching at the indicated time points. The lack of fluorescence recovery indicated that the RNA in the clusters was immobile or in a solid-like state. Scale bar, 1 μm.

FIG. 9F shows representative images of cells expressing 29× GGGGCC or 29× CCCCGG RNA.

FIG. 9G shows the quantification of the number of RNA foci per cell for U-2OS cells expressing 29× GGGGCC or 29× CCCCGG RNA.

FIG. 9H shows that the number of 29× GGGGCC RNA foci increased with the increasing level of RNA expression. The expression levels were controlled by increasing the virus titer.

FIG. 9I shows the quantification of RNA foci at indicated number of GGGGCC repeats.

FIG. 9J shows representative fluorescence micrographs and corresponding quantification of the total volume of foci per cell in U-2OS cells transduced with 12× MS2 tagged RNA with the indicated number of GGGGCC repeats (3×, 9×, 16×, or 29×).

FIGS. 9K and 9L show representative images of GGGGCC RNA punctum at the indicated time points after photobleaching and the corresponding recovery plot.

FIG. 9M shows representative fluorescence micrographs for 29× GGGGCC RNA foci at indicated time points. GGGGCC RNA foci exhibited incomplete recovery upon fluorescence photobleaching.

FIG. 9N shows fluorescence recovery plots for GGGGCC RNA foci with indicated number of repeats. Data are average of n=10 foci at each repeat number.

FIG. 10 shows representative immunofluorescence images illustrating that the 29× GGGGCC RNA foci co-localized with the marker for nuclear speckles (SC-35) but not for Cajal bodies (coilin). The GGGGCC RNA foci also recruited endogenous hnRNP H and MBNL16. Scale bars, 5 μm. Data are representative of three or more independent experiments.

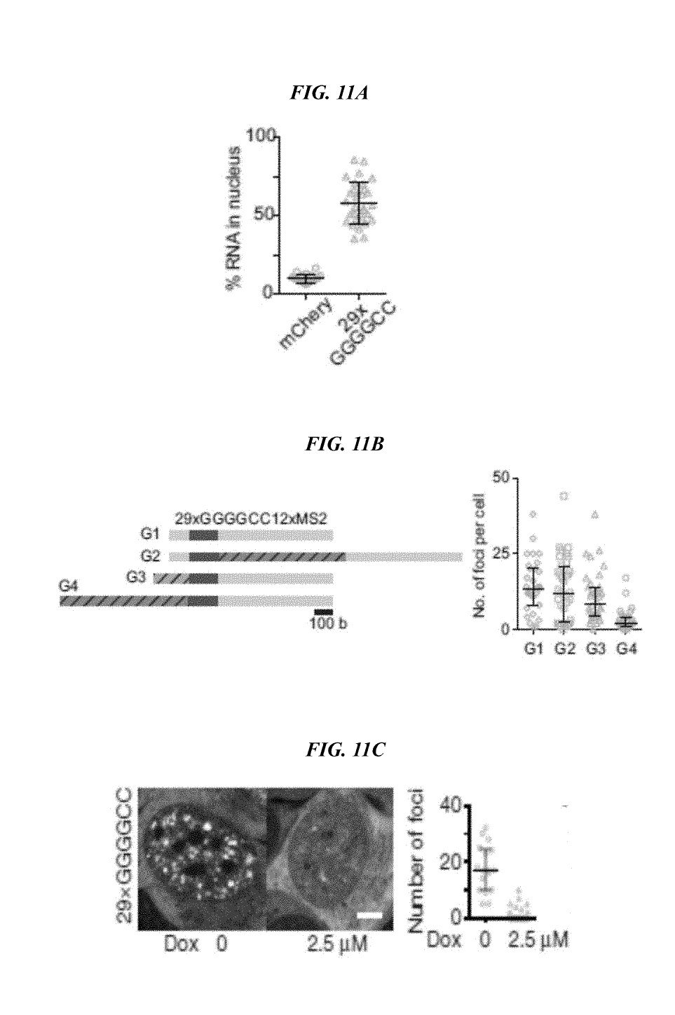

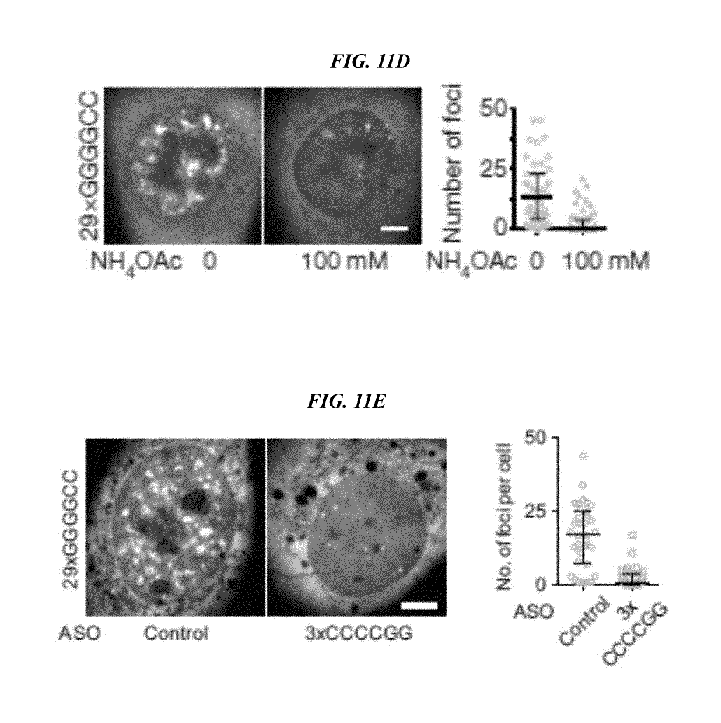

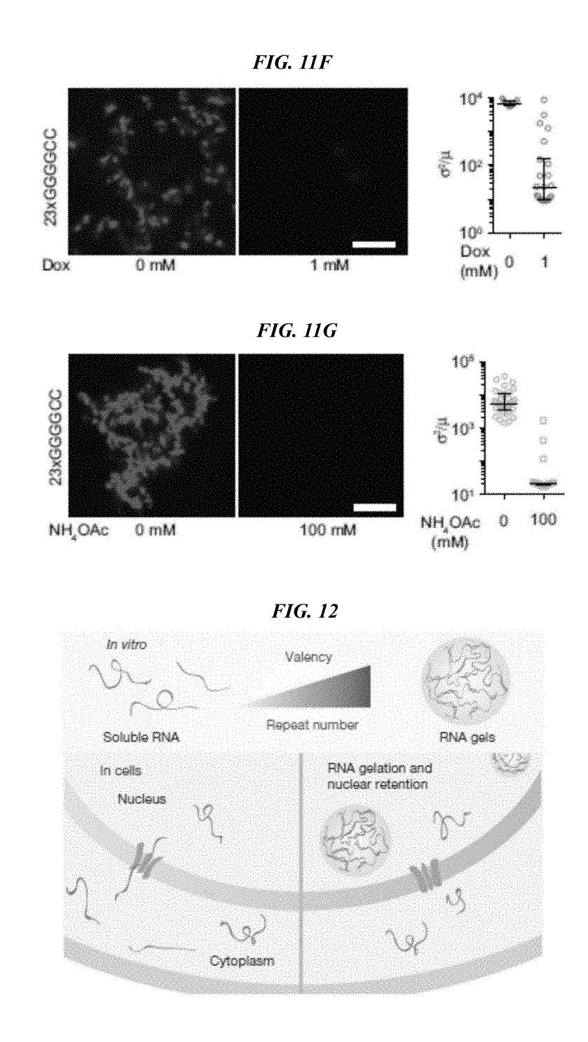

FIG. 11A shows the percentage of 29× GGGGCC RNA retained in the nucleus compared against a control RNA encoding for mCherry.

FIG. 11B shows the effect of flanking sequences on the formation of GGGGCC RNA foci. Construct G1 had 29× GGGGCC repeats with 12× MS2 hairpins (about 0.7 kb) downstream of the repeats for RNA visualization. Incorporation of an approximately 1 kb long sequence (G2) between the 29× GGGGCC repeats and 12× MS2 repeats did not inhibit foci formation. Similarly, RNA foci were observed in construct G3, which had the same 5′ flanking sequence as found in the endogenous locus in intron 1 of c9orf72. However, incorporation of a longer, approximately 1 kb 5′ flanking sequence (G4) inhibited the formation of RNA foci.

FIG. 11C shows representative images and quantification of doxorubicin disrupting 29× GGGGCC RNA foci. Scale bars, 5 μm. Data are median±interquartile range. Data are representative of at least three independent experiments.

FIG. 11D shows representative images and quantification of NH₄OAc disrupting 29× GGGGCC RNA foci. Scale bars, 5 μm. Data are median±interquartile range. Data are representative of at least three independent experiments. The images are of the same cell before and 300 s after treatment.

FIG. 11E shows representative micrographs and quantification of the number of RNA foci per cell. Transfection of U-2OS cells with a 3× CCCCGG ASO disrupted the 29× GGGGCC RNA foci while a control ASO did not.

FIG. 11F shows representative fluorescence micrographs and corresponding quantification of inhomogeneity for 23× GGGGCC RNA in vitro with or without 1 mM doxorubicin.

FIG. 11G shows representative fluorescence micrographs and corresponding quantification of inhomogeneity for 23× GGGGCC RNA in vitro with or without 100 mM NH₄OAc.

FIG. 12 shows a schematic illustrating a model for RNA foci formation in repeat expansion disorders. The repeat expansion sequences form templates for multivalent intermolecular base-pairing, which leads to the formation of RNA foci and the retention of the RNA in the nucleus.

### DETAILED DESCRIPTION OF THE INVENTION

### I. INTRODUCTION

Expansions of short nucleotide repeats produce several neurological and neuromuscular disorders including Huntington's disease, muscular dystrophy, and amyotrophic lateral sclerosis. A common pathological feature of these diseases is the accumulation of the repeat-containing transcripts into aberrant foci in the nucleus. RNA foci, as well as the disease symptoms, only manifest above a critical number of nucleotide repeats. As disclosed herein in the Examples section below, it has been surprisingly found that the RNA foci arise from repeat expansions creating templates for multivalent base-pairing, which causes transcribed RNA to undergo a sol-gel phase transition. Without being bound to a particular theory, it is believed that the sequence-specific gelation is a contributing factor to neurological disease in repeat expansion disorders such as Huntington's disease, muscular dystrophy, and amyotrophic lateral sclerosis.

In one aspect, engineered cells and cell-based assays are provided for detecting the formation of clusters of RNA (e.g., base-pairing mediated clusters of RNA) by RNA transcripts comprising a sequence that is prone to forming clusters of RNA. In some embodiments, these cells and cell-based assays can be used as a screening platform to identify agents that prevent, reduce, or inhibit the formation of clusters of RNA or that dissolve clusters of RNA. Thus, in another aspect, methods of detecting the formation of the clusters of RNA (e.g., base-pairing mediated clusters of RNA) by RNA transcripts comprising a sequence that is prone to clustering, as well as methods of identifying agents that dissolve or inhibit the formation of clusters of RNA, and therapeutic methods using agents that prevent, reduce, or inhibit the formation of clusters of RNA or dissolve clusters of RNA, are provided.

### II. DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document.

The term “a sequence that is prone to forming clusters of RNA,” as used with reference to a polynucleotide, refers to a sequence in a polynucleotide (e.g., an RNA) that forms a template for multivalent intermolecular interactions with other polynucleotides (e.g., other RNAs) having identical or substantially identical template-forming sequences. In some embodiments, the sequence that is prone to forming clusters of RNA is a sequence that comprises repeating patterns of short nucleotide sequences (e.g., repeating patterns of a nucleotide sequence that is 1-8, 2-8, or 2-6 nucleotides in length). In some embodiments, the formation of clusters of RNA is mediated by base pairing.

As used herein, the term “tandem nucleotide repeats” refers to short nucleotide sequences (e.g., 1-8 nucleotides or 2-6 nucleotides in length) that are repeated adjacent to each other multiple times (e.g., 2, 3, 4, 5, 10, 15, 20, 25, 30, 40 or more times) in a polynucleotide sequence. In some embodiments, a tandem nucleotide repeat comprises at least 10, 15, 20, 25, 30, 40 or more adjacent repeated nucleotide sequences.

As used herein, the term “clusters of RNA” refers to clusters, gels, or aggregations of RNA transcripts (e.g., RNA transcripts comprising repeating patterns of short nucleotide sequences) that are formed by multivalent interactions between the RNA transcripts. In some embodiments, the clusters of RNA are formed by multivalent base-pairing interactions between RNAs.

The term “binding motif,” as used with reference to a polynucleotide sequence, refers to a polynucleotide sequence to which a detectable molecule can bind or associate. In some embodiments, the detectable molecule is a detectably labeled molecule (e.g., a coat protein from an RNA phage) and the binding motif comprises a sequence that is recognized and bound by the molecule (e.g., a sequence comprising one or more step loops). In some embodiments, the detectable molecule is a fluorophore or fluorogen and the binding motif comprises a sequence that is recognized and bound by the fluorophore or fluorogen (e.g., an RNA aptamer sequence).

As used herein, the terms “nucleic acid” and “polynucleotide” are used interchangeably. Use of the term “polynucleotide” includes oligonucleotides (i.e., short polynucleotides). This term also refers to deoxyribonucleotides, ribonucleotides, and naturally occurring variants, and can also refer to synthetic and/or non-naturally occurring nucleic acids (i.e., comprising nucleic acid analogues or modified backbone residues or linkages), such as, for example and without limitation, phosphorothioates, phosphoramidates, methyl phosphonates, chiral-methyl phosphonates, 2-O-methyl ribonucleotides, peptide-nucleic acids (PNAs), and the like. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (see, e.g., Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “promoter” refers to regions or sequence located upstream and/or downstream from the start of transcription and which are involved in recognition and binding of RNA polymerase and other proteins to initiate transcription.

The term “heterologous,” as used with reference to a component (e.g., a polynucleotide sequence or a detectable molecule, such as a heterologous protein or a heterologous aptamer) of a cell or as used with reference to two components (e.g., a first polynucleotide sequence and a second polynucleotide sequence), refers to a component that is not naturally occurring in the cell or components that are not naturally associated with each other. For example, in some embodiments, a component (e.g., a polynucleotide sequence or a detectable molecule) originates from a different species as the cell, or, if from the same species, is modified from its original form that occurs in the cell. As another example, in some embodiments, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a different gene in the same species).

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “expression cassette” refers to a nucleic acid construct that, when introduced into a host cell, results in transcription and/or translation of an RNA or polypeptide, respectively.

A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid.

As used herein, an “agent” refers to any molecule, either naturally occurring or synthetic, e.g., peptide, protein, oligopeptide (e.g., from about 5 to about 25 amino acids in length, e.g., about 5, 10, 15, 20, or 25 amino acids in length), small organic molecule (e.g., an organic molecule having a molecular weight of less than about 2500 daltons, e.g., less than 2000, less than 1000, or less than 500 daltons), circular peptide, peptidomimetic, antibody, polysaccharide, lipid, fatty acid, inhibitory RNA (e.g., siRNA or shRNA), polynucleotide, oligonucleotide, aptamer, drug compound, or other compound.

The terms “administer,” “administered,” or “administering” refer to methods of delivering agents, compounds, or compositions to the desired site of biological action. These methods include, but are not limited to, topical delivery, parenteral delivery, intravenous delivery, intradermal delivery, intramuscular delivery, colonical delivery, rectal delivery, or intraperitoneal delivery. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

### III. CELLS AN CELL-BASED ASSAYS FOR DETECTING REPEAT-CONTAINING RNAS

In one aspect, cells (e.g., engineered cells) and live cell reporter assays for detecting or visualizing the formation of clusters of RNA (e.g., base-pairing mediated clusters of RNA) are provided. In some embodiments, an isolated cell or a live cell reporter assay comprises:
a heterologous polynucleotide comprising a promoter operably linked to a polynucleotide for encoding an RNA transcript comprising (i) an RNA sequence comprising a sequence that is prone to forming clusters of RNA and (ii) a binding motif for binding to a detectable molecule; and
a heterologous detectable molecule that binds to the binding motif.

Cells

In some embodiments, the cell is a prokaryotic cell. In some embodiments, the cell is a eukaryotic cell. In some embodiments, the cell is a bacterial or fungal cell. In some embodiments, the cell is a yeast cell, a plant cell, an insect cell, or a mammalian cell. In some embodiments, the cell is a mammalian cell, e.g., a cell from a mouse, rat, human, primate, Chinese hamster, or canine. In some embodiments, the cell is a human cell.

In some embodiments, the cell is a primary cell. In some embodiments, the cell is from brain, nervous tissue, thyroid, eye, skeletal muscle, cartilage, kidney, lung, liver, heart, or bone tissue, or from blood, serum, plasma, or cerebrospinal fluid. In some embodiments, the cell is from a transformed cell line, such as but not limited to a HeLa or U-2 OS (osteocarcoma) cell.

RNA Sequences

In some embodiments, the cell comprises a heterologous polynucleotide comprising one or more RNA sequences comprising a sequence that is prone to forming clusters of RNA. In some embodiments, the sequence that is prone to forming clusters of RNA has a length of at least about 50 nucleotides, e.g., at least 60, 70, 80, 90, 100, 110, 120, 130, 140, 150, 160, 170, 180, 190, 200, 225, 250, 275, 300 or more nucleotides. In some embodiments, the sequence that is prone to forming clusters of RNA comprises a repeating pattern of short nucleotide sequences (e.g., repeating patterns of a nucleotide sequence that is 1-10, 2-8, or 2-6 nucleotides in length). In some embodiments, the sequence that is prone to forming clusters of RNA has a length of at least about 50 nucleotides, e.g., at least 60, 70, 80, 90, 100, 110, 120, 130, 140, 150, 160, 170, 180, 190, 200, 225, 250, 275, 300 or more nucleotides, and further comprises at least 15, 20, 25, 30, 35, 40, 45, 50 or more repeats of a short nucleotide sequence (e.g., a nucleotide sequence that is 1-10, 2-8, or 2-6 nucleotides in length). In some embodiments, the formation of clusters of RNA is mediated by base pairing. In some embodiments, a sequence that is prone to forming clusters of RNA is a polynucleotide sequence that forms multivalent intermolecular interactions with other polynucleotides, e.g., through base pairing or some other type of molecular interaction.

In some embodiments, the RNA sequence that is prone to forming clusters of RNA comprises sequences that form Watson-Crick base pairing (e.g., adenine (A)-thymine (T) or guanine (G)-cytosine (C) interactions), non-canonical base pairing (e.g., interaction between G with U within a secondary structure of RNA), and/or helical stacking (e.g., parallel or antiparallel

A-D/B-C RNA helical stacks; parallel or antiparallel A-B/C-D RNA helical stacks).

In some embodiments, the heterologous polynucleotide encodes an RNA transcript that comprises one or more RNA sequences comprising tandem nucleotide repeats (e.g., multiple nucleotide repeats comprising at least 10, 15, 20, 25, 30, 40 or more adjacent repeated nucleotide sequences). In some embodiments, the RNA sequence comprises at least 5 repeats, e.g., at least 6, at least 7, at least 8, at least 9, at least 10, at least 15, at least 20, at least 25, at least 30, at least 35, at least 40, at least 45, at least 50 repeats, at least 60 repeats, at least 70 repeats, at least 80 repeats, at least 90 repeats, or at least 100 repeats.

In some embodiments, the RNA sequence that is prone to forming clusters of RNA comprises long non-coding RNAs (lncRNAs), long mRNAs, an RNA transcript of a cluster of microRNAs (pri-miRNA), centromeric transcripts, or RNA transcripts, overexpression and aggregation of which are associated with a disease or disorder, such as nucleotide repeat sequences that are associated with repeat expansion disorders (e.g., CUG repeats in myotonic dystrophy 1). In some embodiments, the RNA sequence comprises trinucleotide repeats (also referred to as a triplet repeat). In some embodiments, the trinucleotide repeat sequence is a CAG repeat, a CGG repeat, a GCC repeat, a GAA repeat, or a CUG repeat. In some embodiments, the trinucleotide repeat is a CAG repeat. In some embodiments, the RNA sequence comprises at least 25 trinucleotide repeats (e.g., CAG, CGG, GCC, GAA, or CUG repeats), e.g., at least 26, at least 27, at least 28, at least 29, at least 30, at least 35, at least 40, at least 45, at least 50, at least 60, or at least 70 trinucleotide repeats.

In some embodiments, the RNA sequence comprises tetranucleotide repeats. In some embodiments, the tetranucleotide repeat is a CCUG repeat. In some embodiments, the RNA sequence comprises at least 25 tetranucleotide repeats (e.g., CCUG repeats), e.g., at least 26, at least 28, at least 30, at least 35, or at least 40 hexanucleotide repeats.

In some embodiments, the RNA sequence comprises pentanucleotide repeats. In some embodiments, the pentanucleotide repeat is a AUUCU repeat. In some embodiments, the RNA sequence comprises at least 22 pentanucleotide repeats (e.g., AUUCU repeats), e.g., at least 24, at least 26, at least 28, or at least 30 hexanucleotide repeats.

In some embodiments, the RNA sequence comprises hexanucleotide repeats. In some embodiments, the hexanucleotide repeat is a GGGGCC repeat. In some embodiments, the RNA sequence comprises at least 5 hexanucleotide repeats (e.g., GGGGCC repeats), e.g., at least 6, at least 7, at least 8, at least 9, at least 10, at least 12, at least 14, at least 16, at least 18, at least 20, at least 22, or at least 24, at least 26, at least 28, or at least 30 hexanucleotide repeats.

In some embodiments, the RNA sequence that is prone to forming clusters of RNA, forms such clusters by aggregating a protein (e.g., a Muscleblind RNA-binding protein, or p53 aggregation modulated by RNAs).

Binding Motifs

The heterologous polynucleotide further comprises one or more binding motifs for binding to a detectable molecule that is introduced into the cell. In some embodiments, the binding motif comprises a sequence having a length of at least about 50 nucleotides, e.g., at least 60, 70, 80, 90, 100, 110, 120, 130, 140, 150, 160, 170, 180, 190, 200, 225, 250, 275, 300, 350, 400, 450, 500 or more nucleotides. In some embodiments, the binding motif comprises a sequence having a length of about 50-1000 nucleotides, e.g., about 50-750, 50-500, 100-1000, or 75-500 nucleotides in length.

In some embodiments, the binding motif comprises a polynucleotide sequence that is recognized and bound by an RNA-binding molecule. In some embodiments, the binding motif comprises a polynucleotide sequence that is recognized and bound by a coat binding protein from an RNA phage, e.g., a coat binding protein from the RNA phage MS2, PP7, or Qβ. In some embodiments, the binding motif comprises a hairpin loop or stem loop sequence. In some embodiments, the hairpin loop sequence comprises one or more hairpin loops, e.g., 3, 4, 5, 6, 7, 8, 9, 10, 12, 15, 18 or more hairpin loops. In some embodiments, the binding motif comprises 6, 12, 18, or 24 hairpin loops. Hairpin loop sequences that are recognized by RNA phage coat-binding proteins are known in the art. See, e.g., Lim et al., Nucleic Acids Res, 2002, 30:4138-4144; and Bertrand et al., Mol Cell, 1998, 2:437-445. In some embodiments, the binding motif comprises a hairpin loop sequence comprising 6, 12, 18, or 24 MS2 hairpin loops. In some embodiments, the binding motif comprises a hairpin loop sequence comprising 6, 12, 18, or 24 PP7 hairpin loops. In some embodiments, the binding motif comprises a hairpin loop sequence comprising 6, 12, 18, or 24 Qβ hairpin loops.

In some embodiments, the binding motif comprises a polynucleotide sequence that is recognized and bound by a fluorophore or fluorogen. In some embodiments, the binding motif comprises an RNA aptamer sequence. Polynucleotide sequences, such as RNA aptamer sequences, for binding fluorophores or fluorogens, are known in the art. See, e.g., Dolgosheina et al., WIREs RNA, 2016, 7: 843-851; and Ouellet, Front. Chem., 2016, doi:10.3389/fchem.2016.00029. In some embodiments, the binding motif comprises the sequence of the RNA aptamer Spinach, or a variant or derivative of the Spinach aptamer. See, e.g., Paige et al., Science, 2011, 333:643-646.

Promoters

In some embodiments, the heterologous polynucleotide comprises a promoter. In some embodiments, the promoter and the rest of the sequence in the heterologous polynucleotide are derived from the same species. In some embodiments, the promoter and the rest of the sequence in the heterologous polynucleotide are derived from different species. A promoter may be either eukaryotic or prokaryotic origin.

A promoter may be a constitutive promoter or an inducible promoter. A promoter may also function to direct the specific expression of the heterologous polynucleotide in a specific cell type or a specific location or compartment inside the cell. For example, a promoter may be employed to direct expression of the heterologous polynucleotide in all cellular compartments. Such promoters are referred to herein as “constitutive” promoters and are active under most environmental conditions and states of development or cell differentiation. Alternatively, a promoter may direct expression of the heterologous polynucleotide in a specific location or compartment within the cell (tissue-specific promoters) or may be otherwise under more precise environmental control (inducible promoters). Inducible promoters are activated by an inducing agent, which may be a molecule (e.g., doxycycline, tetracycline, galactose, metal ions, alcohol, or a steroid compound) or an environmental condition (e.g., light, temperature, or pH).

Various types of promoters are known in the art and can be found in, e.g., Qin et al., PLoS One 5:e10611, 2010 and Damdindorj et al., PLoS One 9:e106472. Examples of constitutive promoters include, but are not limited to, human β-actin, human elongation factor-1α, chicken β-actin combined with cytomegalovirus early enhancer, cytomegalovirus (CMV), simian virus 40 (SV40), herpes simplex virus thymidine kinase, UBC, EF1A, PGK, CAG, ubiquitin C promoter, a phosphoglycerate kinase 1 promoter (PGK), T7, Sp6, trp, Ptac, pL, PGK1, Ac5, polyhedrin, TEF1, GDS, CaMV35S, Ubi, H1, and U6. Examples of inducible promoters include, but are not limited to, TRE promoter (tetracycline or doxycycline inducible), lac (IPTG inducible), GAL1 (galactose inducible), T7lac (IPTG inducible), and araBAD (arabinose inducible). In some embodiments, the promoter is a tetracycline-inducible or doxycycline-inducible promoter.

Detectable Molecules

As used herein, a “detectable molecule” is a molecule detectable by spectroscopic, photochemical, biochemical, immunochemical, chemical, magnetic resonance imaging, or other physical means. For example, useful detectable molecules include ³²P, fluorescent dyes, electron-dense reagents, enzymes, biotin, digoxigenin, paramagnetic molecules, and paramagnetic nanoparticles. In some embodiments, the cell comprises one or more heterologous detectable molecules that binds to or associates with the heterologous polynucleotide, e.g., at the binding motif. In some embodiments, the heterologous detectable molecule that binds at the binding motif is a polynucleotide-binding molecule (e.g., an RNA-binding molecule) that further comprises a detectable label. In some embodiments, the heterologous detectable molecule is a coat binding protein from a phage (e.g., from an RNA phage) that binds to a polynucleotide sequence of the binding motif. In some embodiments, the heterologous detectable molecule is a fluorogenic, chromogenic, or otherwise detectable molecule that is able to bind to a polynucleotide sequence of the binding motif.

In some embodiments, the detectable molecule is a coat binding protein from a phage (e.g., from an RNA phage) that comprises a detectable label. In some embodiments, the detectable molecule is a coat binding protein from an RNA phage selected from the group consisting of MS2, PP7, and Qβ. In some embodiments, the detectable molecule is an MS2 coat binding protein. In some embodiments, the detectable molecule is a PP7 coat binding protein.

In some embodiments, the detectable molecule is a fluorogenic, chromogenic, or otherwise detectable molecule that is able to bind to a polynucleotide sequence of the binding motif. In some embodiments, the detectable molecule is a fluorogen or fluorophore that is able to bind to a polynucleotide sequence of the binding motif. In some embodiments, the detectable molecule is 4-hydroxybenzylidene imidazolinone (HBI) or a derivative thereof, such as 3,5-difluoro-4-hydroxybenzylidene imidazolinone (DFHBI), DFHBI-1T, or DFHBI-2T.

In some embodiments, a detectable molecule or detectable label is a molecule or label that produces a readable or detectable signal directly (e.g., a fluorescent protein, an organic fluorophore, or a fluorogen). In some embodiments, a detectable molecule or detectable label is a molecule or label that can be specifically bound by a secondary molecule, which then produces a readable or detectable signal or can be further amplified to produce a readable or detectable signal. In some embodiments, the detectable label is a fluorophore or fluorescent protein.

Examples of fluorescent proteins are well-known in the art, see, e.g., Gert-Jan Kremers et al., J Cell Sci. 124:157, 2011 and Stepanenko et al., Curr Protein Pept Sci. 9:338, 2008. Examples of fluorescent proteins include, but are not limited to, green fluorescent protein (GFP), yellow fluorescent protein (YFP), enhanced blue fluorescent protein (EBFP), azurite, GFPuv, T-Sapphire, Cerulean, mCFP, mTurquoise2, ECFP, CyPet, mKeima-Red, TagCFP, AmCyan1, mTFP1, Midoriishi Cyan, TurboGFP, TagGFP, Emerald, Azami Green, ZsGreenl, TagYFP, EYFP, Topaz, Venus, mCitrine, YPet, TurboYFP, ZsYellowl, Kusabira Orange, mOrange, Allophycocyanin (APC), mKO, TurboRFP, tdTomato, TagRFP, DsRed monomer, DsRed2, mStrawberry, TurboFP602, AsRed2, mRFP1, J-Red, R-phycoerythrin (RPE), B-phycoerythrin (BPE), mCherry, HcRed1, Katusha, P3, Peridinin Chlorophyll (PerCP), mKate (TagFP635), TurboFP635, mPlum, and mRaspberry.

Examples of organic fluorophores include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine and ring-substituted squaraine derivatives (e.g., Seta, SeTau, and Square dyes), naphthalene derivatives (e.g., dansyl and prodan derivatives), coumarin derivatives (e.g., Pacific Blue), oxadiazole derivatives (e.g., pyridyloxazole, nitrobenzoxadiazole, and benzoxadiazole), anthracene derivatives (e.g., anthraquinones, DRAQ5, DRAQ7, and CyTRAK Orange), pyrene derivatives (e.g., cascade blue), oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin).

In some embodiments, a detectable molecule may be a fluorogen, which is not fluorescent itself but becomes fluorescent when it is bound by a specific nucleic acid sequence or nucleic acid structure (e.g., an RNA binding motif as described herein, such as an RNA aptamer sequence (e.g., a Spinach aptamer or a variant or derivative of the Spinach aptamer)). Examples of fluorogens are known in the art and can be found in, e.g., Franzini et al., Org Lett. 10:2935, 2008 and Shibata et al., Chem Commun (Camb) 43:6586, 2009.

A detectable molecule may also be a protein or peptide that can be specifically bound by a secondary molecule, which then produces a readable or detectable signal or can be further amplified to produce a readable or detectable signal. For example, a detectable molecule may be a hexa-histidine peptide, a FLAG peptide, a Myc peptide, or a hemagglutinin (HA) peptide. Each of these peptides can be detected using a specific secondary antibody, e.g., anti-His, anti-FLAG, anti-Myc, or anti-HA antibody. In some embodiments, the secondary antibody may produce a detectable signal directly, i.e., if the secondary antibody is conjugated to a fluorescent protein or organic fluorophore. In some embodiments, the secondary antibody may be further bound by a tertiary antibody, e.g., a tertiary antibody conjugated to horseradish peroxidase (HRP).

### IV. METHODS USING CELLS OR CELL-BASED ASSAYS FOR DETECTING THE FORMATION OF CLUSTERS OF RNA

In another aspect, methods of detecting the formation of clusters of RNA are provided. In some embodiments, the method comprises:
(a) inducing transcription of the RNA sequence comprising a sequence that is prone to forming clusters of RNA (e.g., an RNA sequence comprising tandem nucleotide repeats) in a cell as disclosed herein (e.g., a heterologous polynucleotide comprising a promoter operably linked to a polynucleotide for encoding an RNA transcript comprising (i) an RNA sequence comprising a sequence that is prone to forming clusters of RNA and (ii) a binding motif for binding to a detectable molecule; and comprising a heterologous detectable molecule that binds to the binding motif), thereby forming transcribed RNAs comprising a sequence that is prone to forming clusters of RNA; and
(b) detecting the formation of one or more clusters of RNA in the cell.

In some embodiments, the clusters of RNA are mediated by base pairing.

In some embodiments, the cell is an engineered cell as disclosed herein (e.g., in Section III above).

Inducing Transcription of RNA Sequences

In some embodiments, the method comprises inducing the transcription of RNA sequence comprising a sequence that is prone to forming clusters of RNA (e.g., RNA transcripts comprising tandem nucleotide repeats, e.g., multiple nucleotide repeats comprising at least 10, 15, 20, 25, 30, 40 or more adjacent repeated nucleotide sequences). In some embodiments, transcription is induced in a cell by expressing the polynucleotide comprising the RNA sequence, e.g., under the control of a constitutive promoter or an inducible promoter. In some embodiments, wherein the polynucleotide is expressed under the control of an inducible promoter, expression is induced for a defined period of time, e.g., for at least 12 hours, e.g., at least 24, 36, or 48 hours. In some embodiments, expression is induced for about 12-48 hours, e.g., about 12-36 or 12-24 hours.

In some embodiments, a constitutive promoter is used to drive the expression of the RNA sequence in all cell types or all locations or compartments within a cell. The RNA sequence comprising a sequence that is prone to forming clusters of RNA (e.g., an RNA sequence comprising tandem nucleotide repeats) may comprise a constitutive promoter, such as human (3-actin, human elongation factor-la, chicken (3-actin combined with cytomegalovirus early enhancer, cytomegalovirus (CMV), simian virus 40 (SV40), herpes simplex virus thymidine kinase, UBC, EF1A, PGK, CAG, ubiquitin C promoter, a phosphoglycerate kinase 1 promoter (PGK), T7, Sp6, trp, Ptac, pL, PGK1, Ac5, polyhedrin, TEF1, GDS, CaMV35S, Ubi, H1, or U6.

In some embodiments, an inducible promoter is used to drive the expression of the RNA sequence only in the presence of an inducing agent. The RNA sequence under an inducible promoter may be expressed in specific cell types or specific cellular compartments.

An inducing agent may be a molecule, such as doxycycline, tetracycline, galactose, metal ions, alcohol, or a steroid compound. An inducible promoter may also be activated by environmental conditions, such as light, temperature, or pH. The RNA sequence comprising a sequence that is prone to forming clusters of RNA (e.g., an RNA sequence comprising tandem nucleotide repeats) may comprise a inducible promoter, such as TRE promoter (tetracycline or doxycycline inducible), lac (IPTG inducible), GAL1 (galactose inducible), T7lac (IPTG inducible), or araBAD (arabinose inducible).

Detection and Quantification

In some embodiments, the step of detecting the formation of one or more clusters of RNA by the repeat-containing RNAs comprises detecting the presence of the detectable molecule that binds to the binding motif in the RNA sequence or a detectable signal produced by the detectable molecule.

Methods of detecting and quantifying clusters of RNA formed by RNA transcripts are known in the art and are also described herein, e.g., in the Examples section below. See, e.g., Wojciechowska et al., Hum Mol Genet, 2011, 20:3811-3821; and Weil et al., Trends Cell Biol, 2010, 20:380-390. In some embodiments, clusters of RNA are quantified by measuring a detectable signal (e.g., fluorescence intensity and a size-based threshold to identify RNA clusters). In some embodiments, clusters of RNA are quantified by visual inspection (e.g., using microscopy).

A signal from a directly or indirectly detectable molecule or label can be analyzed, for example, using microscopy (e.g., confocal microscopy, such as spinning disk confocal microscopy, fluorescent microscopy, multiphoton microscopy, or FRAP microscopy); a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays can be automated or performed robotically, and the signal from multiple samples can be detected simultaneously. In some embodiments, the amount of signal can be quantified using an automated high-content imaging system. High-content imaging systems are commercially available (e.g., ImageXpress, Molecular Devices Inc., Sunnyvale, Calif.).

In some embodiments, the detecting step comprises detecting clusters of RNA that exhibit RNA gelation. Characteristics of RNA gelation are described in the Examples section below. For example, in some embodiments, the clusters of RNA exhibit decreased mobility as compared to soluble (non-clustered) RNA transcripts.

In some embodiments, the clusters of RNA that are detected and/or quantified are formed in the nucleus of the cell. In some embodiments, the clusters of RNA that are detected and/or quantified are formed in the cytoplasm of the cell. In some embodiments, the clusters of RNA that are detected and/or quantified are formed in one or more organelles within the cell.

### V. METHODS OF IDENTIFYING AGENTS THAT INHIBIT THE FORMATION OF CELLULAR CLUSTERS OF RNA

In yet another aspect, methods of identifying an agent that dissolves or inhibits the formation of cellular clusters of RNA are provided. In some embodiments, the method comprises:
(a) contacting an agent to a cell or live cell reporter assay as disclosed herein (e.g., a heterologous polynucleotide comprising a promoter operably linked to a polynucleotide for encoding an RNA transcript comprising (i) an RNA sequence comprising a sequence that is prone to forming clusters of RNA and (ii) a binding motif for binding to a detectable molecule; and comprising a heterologous detectable molecule that binds to the binding motif), wherein the cell comprises a plurality of RNA transcripts comprising a sequence that is prone to forming clusters of RNA;
(b) quantifying the amount of clusters of RNA formed by the RNA transcripts in the cell that has been contacted with the agent; and
(c) comparing the amount of clusters formed in (b) with a control value, wherein an amount of clusters of RNA formed in (b) that is less than the control value identifies the agent as an agent that dissolves or inhibits the formation of the clusters of RNA.

In some embodiments, the clusters of RNA that are quantified are formed in the nucleus of the cell. In some embodiments, the clusters of RNA that are quantified are formed in the cytoplasm of the cell. In some embodiments, the clusters of RNA that are quantified are formed in one or more organelles within the cell. In some embodiments, the clusters of RNA are mediated by base pairing.

Agents

Essentially any chemical agent or compound can be tested for its ability to dissolve or inhibit the formation of cellular clusters of RNA. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), Tripos, Inc. (St. Louis, Mo.); and Selleckchem (Houston, Tex.). In some embodiments, the agent is a small molecule, an oligonucleotide, or a protein.

In some embodiments, libraries of small molecules may be screened to identify small molecule agents that may dissolve or inhibit the formation of cellular clusters of RNA. Representative small molecule libraries include, but are not limited to, diversomers such as hydantoins, benzodiazepines, and dipeptides (Hobbs et al., Proc. Nat. Acad. Sci. USA, 90:6909-6913 (1993)); analogous organic syntheses of small compound libraries (Chen et al., J. Amer. Chem. Soc., 116:2661 (1994)); oligocarbamates (Cho et al., Science, 261:1303 (1993)); benzodiazepines (e.g., U.S. Pat. No. 5,288,514; and Baum, C&EN, Jan 18, page 33 (1993)); isoprenoids (e.g., U.S. Pat. No. 5,569,588); thiazolidinones and metathiazanones (e.g., U.S. Pat. No. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., U.S. Pat. No. 6,790,965); amines (e.g., U.S. Pat. No. 6,750,344); phenyl compounds (e.g., U.S. Pat. No. 6,740,712); azoles (e.g., U.S. Pat. No. 6,683,191); pyridine carboxamides or sulfonamides (e.g., U.S. Pat. No. 6,677,452); 2-aminobenzoxazoles (e.g., U.S. Pat. No. 6,660,858); isoindoles, isooxyindoles, or isooxyquinolines (e.g., U.S. Pat. No. 6,667,406); oxazolidinones (e.g., U.S. Pat. No. 6,562,844); and hydroxylamines (e.g., U.S. Pat. No. 6,541,276).

In some embodiments, libraries of oligonucleotides may be screened to identify oligonucleotide agents that may dissolve or inhibit the formation of cellular clusters of RNA. Representative oligonucleotide libraries include, but are not limited to, genomic DNA, cDNA, mRNA, inhibitory RNA (e.g., RNAi, siRNA), and antisense RNA libraries. See, e.g., Ausubel, Current Protocols in Molecular Biology, eds. 1987-2005, Wiley Interscience; and Sambrook and Russell, Molecular Cloning: A Laboratory Manual , 2000, Cold Spring Harbor Laboratory Press. Nucleic acid libraries are described in, for example, U.S. Pat. Nos . 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference, or siRNA libraries, are described in, for example, Downward, Cell, 121:813 (2005) and Akashi et al., Nat. Rev. Mol. Cell Biol., 6:413 (2005). Antisense RNA libraries are described in, for example, U.S. Pat. Nos. 6,586,180 and 6,518,017.

In some embodiments, libraries of proteins may be screened to identify protein agents that may dissolve or inhibit the formation of cellular clusters of RNA. Representative protein libraries include, but are not limited to, peptide libraries (see, e.g., U.S. Pat. Nos. 5,010,175; 6,828,422; and 6,844,161; Furka, Int. J. Pept. Prot. Res., 37:487-493 (1991); Houghton et al., Nature, 354:84-88 (1991); and Eichler, Comb Chem High Throughput Screen., 8:135 (2005)), peptoids (PCT Publication No. WO 91/19735), encoded peptides (PCT Publication No. WO 93/20242), random bio-oligomers (PCT Publication No. WO 92/00091), vinylogous polypeptides (Hagihara et al., J. Amer. Chem. Soc., 114:6568 (1992)), nonpeptidal peptidomimetics with β-D-glucose scaffolding (Hirschmann et al., J. Amer. Chem. Soc., 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT Application No. PCT/US96/10287; and Vaughn et al., Nature Biotechnology, 14:309-314 (1996)), and peptidyl phosphonates (Campbell et al., J. Org. Chem., 59:658 (1994)).

Devices for the preparation of combinatorial libraries are commercially available. See, e.g., 357 MPS and 390 MPS from Advanced Chem. Tech (Louisville, Ky.), Symphony from Rainin Instruments (Woburn, Mass.), 433A from Applied Biosystems (Foster City, Calif.), and 9050 Plus from Millipore (Bedford, Mass.).

In particular embodiments, an agent that dissolves or inhibits the formation of cellular clusters of RNA may be an intercalating agent, which disrupts nucleic acid base pairing by inserting between neighboring nucleic acid bases. Examples of intercalating agents are known in the art and can be found in, e.g., Braila et al., Curr Pharm Des, 2001, 7:1745-1780. In some embodiments, intercalating agents may be polycyclic, aromatic, and/or planar. Examples of intercalating agents include, but are not limited to, acridine, doxorubicin, daunomycin, daunorubicin, dactinomycin, cisplatin, carboplatin, thalidomide, and berberine.

Reference Values

In some embodiments, the extent or amount of clusters of RNA (e.g., base-pairing mediated clusters of RNA) that are formed by RNA transcripts in a cell that has been contacted with the agent is compared to a control or reference value. A variety of methods can be used to determine the reference value for the formation of clusters of RNA. In one embodiment, a reference value is determined by quantifying the extent or amount of clusters of RNA in the cell prior to contacting the cell with the agent. In one embodiment, a reference value is determined by quantifying the extent or amount of clusters of RNA in a population of cells that has not been contacted with the agent. In some embodiments, a reference value is determined by quantifying the extent or amount of clusters of RNA in a cell or population of cells that has been contacted with an agent that is known to dissolve or inhibit the formation of RNA clusters (e.g., doxorubicin).

In some embodiments, an agent is identified as an agent that dissolves or inhibits the formation of clusters of RNA when the extent or amount of clusters of RNA in the cell contacted with the agent is decreased by at least 10%, 15%, 20%, 25%, 30%, 40%, 50%, 60%, 70%, 80%, 90% or more relative to the control or reference value. In some embodiments, the extent or amount of clusters of RNA in the cell is quantified after the cell has been incubated with the agent for a period of time (e.g., at least about 15 minutes, at least about 30 minutes, at least about 45 minutes, at least about 1 hour, or longer).

Methods of detecting and quantifying clusters of RNA formed by RNA transcripts are known in the art and are also described herein, e.g., in Section IV above and in the Examples section below.

Optimization of Agents Identified in Screen

In some embodiments, after agents that are identified as candidate agents for dissolving or inhibiting the formation of nuclear foci by repeat-containing RNAs, compound optimization is conducted. In some embodiments, an agent is optimized in order to improve the agent's biological and pharmacological properties. In some embodiments, to optimize a selected-for agent or compound, structurally related analogs are chemically synthesized to systematically modify the structure of the initially-identified agent or compound.

For chemical synthesis, solid phase synthesis can be used for compounds such as peptides, nucleic acids, organic molecules, etc. In general, solid phase synthesis is a straightforward approach with excellent scalability to commercial scale. Techniques for solid phase synthesis are described in the art. See, e.g., Seneci, Solid Phase Synthesis and Combinatorial Technologies (John Wiley & Sons 2002); Barany & Merrifield, Solid-Phase Peptide Synthesis, pp. 3-284 in The Peptides: Analysis, Synthesis, Biology, Vol. 2 (E. Gross and J. Meienhofer, eds., Academic Press 1979).

Typically, optimization involves the use of in vitro and in vivo screens (e.g., in an appropriate animal model, e.g., a mammal such as a mouse, rat, or monkey) to assess the biological, pharmacokinetic, and pharmacodynamic properties of the agents or compounds, such as oral bioavailability, half-life, metabolism, toxicity, pharmacokinetic profile, and pharmacodynamic activity. See, e.g., Guido et al., Combinatorial Chemistry & High Throughput Screening, 2011, 14:830-839.

In some embodiments, an agent that is identified as dissolving or inhibiting the formation of clusters of RNA (e.g., base-pairing mediated clusters of RNA) by repeat-containing RNAs, or a structurally related analog thereof, is used for the preparation of a pharmaceutical composition for use in the treatment of a repeat expansion disorder. Typically, the pharmaceutical composition will comprise the agent (e.g., the agent identified by a screening method described herein or a structurally related analog thereof) and one or more pharmaceutically acceptable carriers and/or pharmaceutically acceptable excipients. As used herein, “pharmaceutically acceptable carrier” or “pharmaceutically acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Compositions comprising such carriers are formulated by well-known conventional methods (see, for example, Remington, The Science and Practice of Pharmacy, 22^{nd} edition, Allen, Lloyd V., Jr., ed., Pharmaceutical Press, 2013).

### VI. THERAPEUTIC METHODS

In still another aspect, methods of treating a subject having a disease characterized by clusters of RNA (e.g., a disease characterized by base-pairing mediated clusters of RNA) are provided. In some embodiments, the method comprises administering to the subject an agent that inhibits or dissolves the formation of clusters of RNA by RNA transcripts comprising a sequence that is prone to forming clusters of RNA (e.g., base-pairing mediated clusters of RNA by RNA transcripts comprising tandem nucleotide repeats), or a pharmaceutical composition comprising the agent; thereby treating the subject.

In some embodiments, the disease is a disease that is caused by repeat expansions (e.g., trinucleotide repeat expansions, tetranucleotide repeat expansions, pentanucleotide repeat expansions, or hexanucleotide repeat expansions). In some embodiments, the disease is Huntington's disease, Huntington disease-like 2 (HDL2), myotonic dystrophy, spinocerebellar ataxia, spinal and bulbar muscular atrophy (SBMA), dentatorubral-pallidoluysian atrophy (DRPLA), amyotrophic lateral sclerosis, frontotemporal dementia, Fragile X syndrome, fragile X mental retardation 1 (FMR1), fragile X mental retardation 2 (FMR2), Friedreich's ataxia (FRDA), fragile X-associated tremor/ataxia syndrome (FXTAS), myoclonic epilepsy, oculopharyngeal muscular dystrophy (OPMD), or syndromic or non-syndromic X-linked mental retardation. In some embodiments, the disease is Huntington's disease. In some embodiments, the disease is amyotrophic lateral sclerosis. In some embodiments, the disease is a form of spinocerebellar ataxia (e.g., SCA1, SCA2, SAC3/MJD, SCA6, SCAT, SCAB, SCA10, SCA12, or SCA17). In some embodiments, the disease is a form of myotonic dystrophy (e.g., myotonic dystrophy type 1 or myotonic dystrophy type 2).

In some embodiments, the agent is a small molecule, an oligonucleotide, a protein, or a combination thereof. In some embodiments, the agent is a small molecule. In some embodiments, the agent is an oligonucleotide. In some embodiments, the agent is an intercalating agent. In some embodiments, the agent is an agent identified according to a method described herein (e.g., in Section V above) or a structurally related analog of such agent.

The agents or pharmaceutical compositions are administered in a manner compatible with the dosage formulation, and in such amount as will be therapeutically effective. The term “therapeutically effective amount” refers to that amount of an agent (e.g., a compound or pharmaceutical composition as described herein) being administered that will treat to some extent a disease, disorder, or condition, e.g., relieve one or more of the symptoms of the disease, i.e., infection, being treated, and/or that amount that will prevent, to some extent, one or more of the symptoms of the disease (e.g., repeat expansion disorder), that the subject being treated has or is at risk of developing. In some embodiments, a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. The size of the dose will also be determined by the existence, nature, and extent of any adverse side-effects that accompany the administration of a particular compound in a particular patient. Determination of the proper dosage for a particular situation is within the skill of the practitioner. Frequently, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

### VII. EXAMPLES

The following examples are offered to illustrate, but not to limit, the claimed invention.

### Example 1

### General Methods

Cloning

CAG and GGGGCC repeats were cloned via sequential repeat directed elongation as described in, e.g., Scior et al., BMC Biotechnol. 11:87, 2011, in a modified pBluescript vector. Inserts were verified via sequencing from both ends (up to 700 bp read length for CAG/CTG repeats and up to 200 bp read length for GGGGCC repeats) and by verifying the insert size by restriction digestion. All cloning and amplification were performed in Escherichia coli Stbl3 cells (Invitrogen) grown at 30° C. For synthesizing the mammalian expression constructs, repeats were cut directly from the cloning plasmids and ligated at the compatible restriction sites in a modified lentiviral expression vector with tetracycline-inducible expression promoter. It was observed that the purified plasmids formed higher-order complexes when stored for prolonged periods (>1 month) at 4° C. or −20° C. Stored plasmids when re-transformed in bacteria often resulted in significant truncations in the repeat region. To avoid such re-transformation-associated repeat truncations, a bacterial stock of each plasmid was maintained. The plasmid DNA was freshly purified for each cloning/transfection or RNA transcription experiment, and the sequence was verified as described above.

RNA Transcription and Gelation

Repeat-containing RNA was transcribed using a T7 or T3 MegaScript kit (Ambion) according to the manufacturer's recommendation. Template DNAs up to 200 bases long (for 10×CAG, 20×CAG, 10×CUG, 20×CUG, 3×GGGGCC, 5×GGGGCC, and 23×GGGGCC) were purchased from Integrated DNA Technologies as single-stranded DNA oligonucleotides. Complementary strand was synthesized by using a single complementary primer and a standard polymerase chain reaction kit (Advantage GC 2 PCR Kit, Clontech). The double-stranded DNA thus generated was gel purified and used as a template for transcription reactions. Longer templates were obtained by either PCR amplification from plasmids (31×CAG, 47×CAG, 31×CUG, 47×CUG) or by restriction digestion of the repeat-containing vectors (66×CAG, 66×CUG) upstream and downstream of the repeat region. For synthesis of fluorescently labelled RNA, transcription reactions were doped with Cy3-UTP or Cy5-UTP (Enzo Lifesciences). Free nucleoside 5′-triphosphates were removed using lithium chloride precipitation or an RNA purification kit (Zymo Research). Similar results were obtained in both purification schemes. The size of the RNA products was verified using denaturing agarose gel electrophoresis. RNAs were resuspended in water, and either used immediately or aliquoted and flash-frozen in liquid nitrogen and stored at −80° C. for up to 1 month.

For phase separation/gelation assays, RNAs were diluted to concentrations of 0.5 ng/μL to 0.5 μg/μL in 10 mM Tris pH 7.0, 10 mM MgCl₂, 25 mM NaCl buffer, unless indicated otherwise. Nuclease-free buffer stocks were purchased from Ambion. RNA was denatured at 95° C. for 3 minutes and cooled down at 1-4° C. per minute to 37° C. final temperature in a thermocycler and imaged immediately. Samples were visualized using a custom spinning disk confocal microscope (Nikon Ti-Eclipse equipped with a Yokogawa CSU-X spinning disk module) using a×100, 1.49 numerical aperture oil immersion objective and an air-cooled EM-CCD (electron multiplying charge-coupled device). The extent of phase separation/gelation was quantified by the index of dispersion (σ²/μ) of fluorescence intensity per pixel (pixel size 83 nm×83 nm). Briefly, variance in the fluorescence intensity per image was determined, and normalized to the mean fluorescence intensity in the solution phase of the RNA. For dilute solution (<10% of imaging area occupied with clusters), this parameter reports the extent of inhomogeneity in the sample. At least 20 independent imaging areas (about 1,800 μm² each) were analyzed for each condition to achieve a representative measure across the sample. Each datum point in the bar graphs represents one imaging area. Data shown are representative of three or more independent replicates, across two or more independent RNA preparations.

For antisense DNA-mediated repression of RNA phase separation, 47×CAG RNA (200 ng/μL or 2.4 μM) was incubated with the ASO at 20 μM final concentration, followed by heat denaturation and annealing as described above. Doxorubicin was purchased from Cell Signaling Technology (catalogue number 5927). For in vitro experiments, doxorubicin was added to pre-formed RNA clusters, and samples were incubated at 37° C. for 1 hour. Alternatively, doxorubicin and RNA were pre-mixed at indicated concentrations before annealing. Similar results were obtained in both cases.

For FRAP experiments, RNA clusters were prepared as described above. RNA clusters were allowed to settle on to the glass surface for about 15 minutes. A region of about 1 μm² was photobleached using a 405 nm laser modulated by a Rapp UGA-40 photo targeting unit and the fluorescence recovery was monitored over time. The fluorescence recovery was fitted to the equation I=A−I₀ exp(−t/τF_{RAP}), and time constant, τ_{FRAP}, was determined.

DNA Phase Separation

DNA oligonucleotides were purchased from Integrated DNA Technologies. Spermine hydrochloride (Sigma) was resuspended in water and pH was adjusted to 7.5. DNA was heat denatured at 90° C. for 2 minutes to melt secondary structure, incubated on ice for 2 minutes, and used immediately for phase separation assays. Phase separation was trigged by adding spermine to the DNA solution. All phase separation assays were performed in 10 mM Tris pH 7.0 buffer with the indicated amounts of DNA and salts. DNA clusters were visualized using standard bright-field or confocal microscopy as described above. To prevent DNA droplets from fusing onto the glass surface, coverslips were passivated with polyethylene glycol. FRAP experiments and analysis were performed as described above.

Cell Culture and Imaging

U-2OS cells, authenticated by STR profiling, were purchased from the University of California, San Francisco, Cell Culture Facility. A monoclonal U-20S cell line stably expressing Tet-On 3G transactivator protein (Clontech) and a tandem-dimeric MS2 hairpin binding protein tagged with enhanced YFP (MS2CP-YFP) was generated via sequential lentiviral infection and selection. This stable cell line was transduced with repeat-containing plasmids under doxycyclinetetracycline-inducible promoter. Cells were maintained in DMEM with 10% (v/v) tetracycline-free fetal bovine serum (Clontech) and 1× penicillin-streptomycin-glutamine cocktail (Gibco). Cell lines were tested for mycoplasma contamination using a standard PCR kit (LookOut Mycoplasma PCR Detection Kit, Sigma) and verified routinely by live-cell DNA staining.

RNA expression was induced by adding 1,000 ng/mL doxycycline for 12-48 hours, or as indicated. Before imaging, the culture medium was replaced with DMEM with 25 mM HEPES pH 7.5 or FluoroBrite DMEM (Invitrogen) with serum and antibiotics as listed above. For long-term imaging (>2 hours), cells were placed in a live-cell imaging chamber supplemented with 5% CO₂. Cells were imaged using a spinning disk confocal microscope (Nikon Ti-Eclipse equipped with a Yokogawa CSU-X spinning disk module) using a×100, 1.49 numerical aperture oil immersion objective and an air-cooled EM-CCD. For each experimental condition, at least 30 randomly chosen cells were imaged and analyzed. Each datum point in the bar graphs represents one cell. Data shown are representative of three or more independent replicates.

ATP depletion was achieved by rinsing cells twice in DMEM without glucose (Gibco), followed by incubation for 10 minutes in the ATP depletion medium (DMEM without glucose with 1% (v/v) dialyzed FBS (Gibco), 10 mM sodium azide and 6 mM 2-deoxy-D-glucose). Doxorubicin (stock, 10 mM in dimethylsulfoxide (DMSO)) was diluted to the desired concentration in cell culture medium and added to cells pre-induced with doxycycline for 24 hours. Cells were incubated with doxorubicin or an equivalent dilution of DMSO only as control, for 2 hours, and imaged as described above. Ammonium acetate (stock, 5 N) was diluted to 200 mM in cell culture medium. This intermediate dilution (2×) was added to cells pre-induced with doxycycline for 24 hours to achieve a final concentration of 100 mM of ammonium acetate. Cells were incubated in this medium for 10 minutes at 37° C. Normal cell culture medium was replaced after treatment, and cells were imaged immediately or 1 hour after medium replacement. For treatment with ASO, cells pre-induced with doxycycline for 48 hours were transfected with 100 nM final concentration of ASO using either Lipofectamine RNAiMAX (Invitrogen) or TranslT-Oligo Transfection Reagent (Mirius Bio) according to the manufacturers' recommended protocols. Similar results were obtained with both transfection reagents. Cells were imaged 12 hours after transfection.

Analysis of RNA Foci

A fluorescence-intensity and size-based threshold was used to identify RNA foci. Briefly, U-2OS cells expressing the RNA of interest together with MS2CP-YFP were imaged using a spinning disk confocal microscope, and 0.3 μm Z-stacks were acquired. To account for variability in MS2CP-YFP expression levels, a cell-intrinsic intensity threshold was used for foci identification. The nuclei were manually segmented, and the mean YFP fluorescence intensity in the nucleus was manually determined. RNA foci were identified using the FIJI 3D Objects Counter plugin, with an intensity threshold as 1.6x the mean fluorescence intensity in the nucleus of the cell, and a size cut-off of more than 50 adjoining pixels (pixel size, 83 nm×83 nm). This algorithm faithfully identified the foci. This method was used to determine the number, volume, surface area, and the fluorescence intensity of the foci. Various metrics such as total number of foci per cell, total volume of foci per cell, coefficient of dispersion (σ²/μvariance/mean), and integrated intensity of foci were compared and yielded similar results. The number of foci per cell, and the total volume occupied by the foci per cell, were chosen as the parameters of choice to quantify the extent of foci formation. Statistical significance was analyzed using unpaired, two-tailed Mann-Whitney U-tests. For this analysis, the numbers of foci per cell in each experiment were assumed to be symmetrically distributed about the median.

Quantification of RNA Copy Number

To quantify the copy number of RNA in cells, two alternative approaches were used. First, NanoString, a proprietary PCR-free RNA quantitation platform, was used to determine that, under the highest induction conditions, the copy number of 47×CAG RNA is about ten times that of GAPDH or β-actin RNA, or about 8,800±1,500 copies per cell (n=3 independent experiments). Second, single-molecule FISH was used to obtain quantitative RNA localization information. Fluorescent probes against the MS2 hairpin loop region were designed, such that the 12×MS2 tag could accommodate a maximum of 32 fluorescently labelled probes. For cells expressing low levels of MS2-tagged control RNAs such as mCherry or 5×CAG RNA, isolated fluorescent spots that exhibited a uniform distribution of intensities, probably arising from single RNA molecules, were observed. Similarly, in the cytoplasm of cells expressing 47×CAG or 29×GGGGCC RNA, isolated RNA spots with a similar uniform distribution of fluorescence intensities were observed (see, e.g., FIGS. 4Q and 4R). This intensity value was ascribed as corresponding to that of a single RNA. Fluorescence spots and corresponding intensities were quantified using the ImageJ Spot Counter plugin. The approximate RNA copy number in each cell was then calculated by dividing the total fluorescence intensity of the cell by the fluorescence intensity of a single RNA, after background subtraction. By this method, it was determined that, under maximal induction conditions leading to RNA foci formation, the copy number of 47×CAG was 13,000±7,000 copies per cell (mean±s.d., n=24 cells), for 29×GGGGCC RNA 2,500±1,800 copies per cell (n=30 cells), and for the control cells expressing mCherry was 20,000±7,000 copies per cell (n=21 cells). The fraction of RNA retained in the nucleus was determined by dividing the fluorescence intensity in the nucleus of the cell by the total fluorescence of the cell.

FRAP Experiments and Data Analysis

To assess the dynamicity of RNA foci, FRAP experiments by bleaching MS2CP-YFP protein were performed. Previous studies have shown that the dimeric MS2CP-YFP is attached with high affinity to the MS2 hairpin sequence and does not dissociate during the observation timescales of a few minutes, see, e.g, Shav-Tal, et al., Science 304:1797, 2004, and that the fluorescence recovery of MS2CP-YFP can be used to report on the RNA dynamics. To monitor exchange of RNA between foci and the nucleoplasm, an entire punctum, typically a few micrometres in size, was photobleached and the fluorescence recovery was monitored by time-lapse imaging. To examine internal turnover, relatively large puncta were manually selected and a region about 1μm in diameter was photobleached. The fluorescence intensity of the bleached region was normalized and corrected for photobleaching using previously described methods, see., e.g., Phair et al., Methods Enzymol. 375:393, 2004. To determine fluorescence relaxation time, the recovery curves were fitted to the equation I=A−I₀ exp(−t/τ_{FRAP}), where A and I₀ are also fit parameters.

RNA FISH and Immunofluorescence

For RNA FISH in U-2OS cells, cells expressing the desired RNA (induced for 24 hours) were fixed with 2% paraformaldehyde for 10 minutes at room temperature and permeabilized by overnight incubation in 70% ethanol at 4° C. Alternatively, cells were fixed and permeabilized by incubation for 10 minutes in methanol with 10% (v/v) acetic acid. Similar results were obtained with both fixation protocols. Fixed and permeabilized cells were either used immediately, or stored in the permeabilization medium at −20° C. until needed. RNA was detected using Cy3-labelled DNA oligonucleotides designed against the MS2-hairpin sequence.

Hybridization and wash buffers were purchased from Biosearch Technologies and used according to the manufacturer's protocol. For immunofluorescence detection of proteins, methanol-fixed cells were stained using antibodies against muscleblind-like-1 (MBNL1, Abcam, ab45899), hnRNP H (Abcam, ab10374), SC-35 (Abcam, ab11826), coilin (ab87913), fibrillarin (Abcam, ab5821), PML (Abcam, ab179466), and a corresponding Alexa Fluor 647-labelled secondary antibody (Invitrogen A-21236 or Invitrogen A-21244). Samples were co-stained with an anti-green fluorescent protein (GFP) booster antibody (GBA488, Bulldog Bio) to visualize RNA foci. After labelling, samples were mounted in Prolong Gold antifade medium (Thermo Scientific) and imaged using confocal microscopy as described above.

DM1 Fibroblasts

DM1 fibroblasts were obtained from the Coriell Institute (catalogue numbers GM03132 and GM03987). Control fibroblasts (Hs27) were obtained from the University of California, San Francisco, Cell Culture Facility. These cell lines were used without further validation. Cells were maintained in DMEM with 10% (v/v) fetal bovine serum (Clontech) and 1× penicillin-streptomycin-glutamine cocktail (Gibco). To detect RNA foci, RNA FISH was performed as described above using an 8×CAG oligonucleotide labelled with Atto647N or using a pool of 48 oligonucleotide probes designed against the wild-type DMPK allele obtained as a pooled library from Biosearch Technologies. To disrupt RNA foci, cells were incubated for 24 hours with 2 μM doxorubicin or an equivalent dose of DMSO-only control. Total volume and the number of RNA foci were quantified using the ImageJ 3D Objects Counter plugin, with an empirically determined fluorescence threshold.

### Example 2

### Repeat-Containing RNAs Form Gels In Vitro

To examine whether repeat-containing RNAs assemble into large clusters, fluorescently labelled RNAs containing 47 triplet repeats of CAG (47× CAG) or CUG (47× CUG) were synthesized. As controls, RNAs of equivalent length (about 250 bases), but with arbitrary sequences with 30-75% GC content, and RNAs with scrambled sequences but with identical base composition as 47× CAG and 47× CUG were used. Upon annealing, the 47× CAG and 47× CUG RNAs formed micrometer-sized spherical clusters, while the control RNAs remained soluble (FIGS. 1B-1D). Clusters were observable at RNA concentrations as low as 25 nM (FIG. 1E). These clusters were enriched more than 100-fold in the RNA compared with the solution phase and contained nearly half of the RNA in the reaction (FIG. 1F). The clusters were dissolved by RNase A, but not proteinase K or DNase I (FIG. 1G and 1H), confirming that clustering was not mediated by protein or DNA contaminants. RNA clustering required Mg²⁺ and was inhibited by monovalent cations such as Na⁺ (FIGS. 1I and 1J), suggesting that, besides multivalent base-pairing, electrostatic interactions also play a prominent role in RNA clustering.

Consistent with valency dependence (i.e., molecules that form multivalent interactions show abrupt phase transitions with increasing valency of interaction), it was found that the formation of CAG/CUG RNA clusters occurred only with more than 30 triplet repeats (FIG. 1K). The intermolecular interactions between the transcripts potentially could be competed out by shorter complementary antisense oligonucleotides (ASO). Indeed, a 6× CTG ASO prevented clustering of 47× CAG RNA, while control oligonucleotides did not (FIG. 1L). Collectively, these experiments indicate that intermolecular base-pairing interactions in the CAG/CUG-repeat region can lead to the coalescence of RNAs into micrometer-sized clusters.

### Example 3

### Physical Properties of the CAG/CUG RNA Clusters

The spherical shapes of RNA clusters (i.e., aspect ratio 1.05±0.1, mean±s.d., n=214) are characteristic of polymers undergoing liquid-liquid phase separation. Molecules within the liquid phase are mobile and undergo fast internal rearrangement. However, fluorescence recovery after photobleaching (FRAP) experiments revealed little or no fluorescence recovery over about 10 minutes, indicating that RNA in the clusters was immobile (FIGS. 2A and 2B). It was hypothesized that these RNAs initially phase-separate into spherical liquid-like droplets, but then rapidly become crosslinked into gels because of increasing intermolecular base-pairing. Consistent with this idea, occasional (about 1% of clusters) incomplete fusion events were observed where two droplets solidified before relaxation to a single spherical geometry (FIG. 2C). As further evidence for this model, it was found that single-stranded DNA, in the presence of polyvalent cations, formed liquid-like droplets and that incorporation of multivalent base-pairing sites progressively imparted solid-like properties to the DNA droplets (FIGS. 3A-3D). In summary, multivalent base-pairing interactions lead to the gelation of CAG/CUG-repeat-containing RNAs in vitro at a similar critical number of repeats as observed in diseases.

### Example 4

### CAG-Repeat RNAs Phase-Separate in Cells

A live-cell reporter assay in U-2OS cells was established to visualize repeat-containing RNAs and determine whether they form aberrant nuclear foci. For this purpose, the RNA was tagged with 12× MS2-hairpin loops (see, e.g., Bertrand et al., Mol. Cell 2:437, 1998) and co-expressed yellow fluorescent protein (YFP)-tagged MS2-coat binding protein (MS2CP-YFP) (FIG. 4A). Multiple stop codons were incorporated upstream of the repeats to minimize translation of repeat-containing proteins (FIG. 4A). Upon induction of 47× CAG or 120× CAG RNA transcription, numerous nuclear foci appeared as early as 1 hour after induction (FIGS. 4B-4E). The number of foci per nucleus increased with higher levels of RNA induction (FIG. 4F). In contrast, 5× CAG (FIGS. 4B and 4O) or control RNAs with coding or non-coding sequences did not form nuclear puncta (FIGS. 4P and 4Q) when expressed at similar levels (about 10,000 copies per cell; FIGS. 4R and 4S). The formation of foci did not induce discernible cell death or impede cell growth over a 7-day period after induction (FIG. 4T). Since repeat expansion disorders take years to manifest in patients, short-term toxicity in cells is not necessarily expected.

The CAG RNA nuclear foci exhibited liquid-like properties. For example, two or more foci could fuse with one another (FIGS. 5A and 5B), a hallmark of liquid-like behavior. Upon photobleaching, nuclear foci also exhibited near-complete fluorescence recovery (83±13% recovery, time constant τTFRAP=81±24 s, mean±s.d., n=5 foci), indicating that the RNA can move into and out of the foci (FIGS. 5C and 5D). Upon photobleaching a portion of a 47× CAG RNA punctum, the fluorescence recovered rapidly (τ_{FRAP}=18±5 s, mean±s.d., n=5 foci) (FIGS. 5E and 5F), suggesting that RNA within the foci can undergo internal rearrangement. Thus, unlike their solid-like behavior in vitro (FIG. 2A), CAG RNA foci in cells display liquid-like properties. It was hypothesized that the increased dynamicity might arise from specialized proteins (for example, helicases) in the nucleoplasm that remodel RNA base-pairing. Consistent with this hypothesis, depletion of cellular ATP substantially reduced fluorescence recovery of the RNA foci after photobleaching (23±7% recovery, mean±s.d., n=7; FIG. 5D).

Similar to the endogenous foci in patient-derived fibroblasts (see, e.g., Urbanek et a., Biochim. Biophys. Acta 1862:1513, 2016), the induced RNA foci co-localized with the SC-35 marker for nuclear speckles (FIGS. 6A and 6B), non-membranous bodies that are enriched in pre-messenger RNA (pre-mRNA) splicing factors. The foci also recruited endogenous muscleblind-like-1 (MBNL1) protein (FIG. 6C), sequestration of which has been implicated in CAG/CUG-repeat-containing RNA-mediated pathogenicity (see, e.g., Miller et al., EMBO J. 19:4439, 2000) and Li et al., Nature 453:1107, 2008). Using fluorescence in situ hybridization (FISH), it was found that about 50% of the 47× CAG RNA was retained in the nucleus, compared with <10% of control RNAs (FIGS. 4Q-4S and FIGS. 6D and 6E). Thus, CAG repeats cause the RNA to be retained in the nucleus within liquid-like bodies that sequester splicing factors.

### Example 5

### Inhibitors of RNA Gelation Disrupt Foci

Perturbations that prevent RNA gelation in vitro may also affect the stability of RNA foci in cells. In vitro, RNA gelation is inhibited by monovalent cations (FIGS. 1I and 1J and FIG. 7A). To test the effect of monovalent cations in cells, ammonium acetate which readily permeates into cells and does not perturb intracellular pH3 was used. Strikingly, addition of 100 mM ammonium acetate led to the disappearance of 47× CAG RNA foci within minutes (FIGS. 7B and 7C). Interestingly, nuclear speckles also were disrupted by ammonium acetate, suggesting that the assembly of these ribonucleoprotein bodies depends upon ionic interactions as well (FIGS. 7D and 7E). It was also found that the disruption of nuclear speckles with tautomycin also disrupts the 47× CAG RNA foci (FIGS. 7D and 7E).

Agents that might specifically disrupt the base-pairing in RNA foci without dissolving nuclear speckles were tested. Transfection of an 8× CTG ASO reduced the number and size of 47× CAG foci compared against control oligonucleotides (FIGS. 7F and 7G). ASO may disrupt cellular RNA foci either by inhibiting intermolecular base-pairing or by degrading RNA via the RNase H machinery. To specifically perturb base-pairing interactions, doxorubicin, a nucleic acid intercalator, was used. Doxorubicin blocked the formation of CAG RNA gels in vitro and potently dissolved the 47× CAG nuclear foci in cells (FIGS. 7H-7J) without disrupting nuclear speckles (FIG. 7D). The concentration of doxorubicin required to disrupt RNA foci in cells (2.5 μM) was lower than that needed for the in vitro experiments (about 1 mM), potentially because of the aid of cellular proteins that unwind RNA base-pairing and facilitate doxorubicin intercalation. Further, it was also tested whether doxorubicin can alleviate RNA foci derived from an endogenous locus. To this end, fibroblasts derived from patients with myotonic dystrophy type 1 (DM1) with a CTG expansion in the DMPK gene, which exhibit RNA foci containing multiple DMPK transcripts (FIGS. 8A-8C), were used. Treatment with doxorubicin (2 μM for 24 hours) substantially reduced the number as well as total volume of foci per cell (about 65% and about 85% decrease, respectively; FIGS. 8D and 8E). In summary, agents that inhibit gelation of purified repeat-containing RNAs in vitro also disrupt RNA foci in cells.

### Example 6

### ALS/FTD-Linked GGGGCC Repeats Form Gels

Besides the canonical Watson-Crick base-pairing, nucleic acids can also form Hoogsteen base pairs such as in G-quadruplexes. The GGGGCC repeat in the C9orf7 2 locus associated with ALS/FTD was found to form G-quadruplexes in vitro and in vivo (see, e.g., Conlon et al., eLife 5:345, 2016 and Reddy et al., J. Biol. Chem. 288:9860, 2013). A single G-quadruplex can bring up to four RNA strands together, but a GGGGCC repeat expansion could potentially give rise to multimolecular RNA complexes (FIG. 9A). Indeed, while the 3× GGGGCC RNA is largely soluble, it was found that 5× GGGGCC RNA formed spherical clusters in vitro (FIG. 9B). Longer repeats (10× and 23×) formed an interconnected mesh-like network of aggregated RNA (FIG. 9B). In contrast, 23× CCCCGG RNA, which can form multivalent Watson-Crick base-pairing but not G-quadruplexes, was soluble (FIG. 9C). Similar to the CAG/CUG RNA, GGGGCC RNA clusters exhibited solid-like properties, and clustering was inhibited by monovalent cations (FIGS. 9D and 9E).

Cellular expression of 29× GGGGCC, but not 29× CCCCGG, RNA resulted in the formation of nuclear puncta in a dose-dependent manner (FIGS. 9F-9H). The number of foci per cell increased with the number of repeats (FIGS. 9I and 9J). The threshold number of repeats for disease onset (>23× GGGGCC4) is similar to the repeat length in which most cells exhibit foci (16-29× GGGGCC). This GGGGCC repeat number is higher than that required for RNA gelation in vitro, possibly because of cellular proteins that may unfold G-quadruplexes (see, e.g., Guo et al., Science 353:aaf5371, 2016). Interestingly, the 29× GGGGCC RNA foci, as well as those of shorter length, exhibited incomplete FRAP recovery (37±20% recovery, mean±s.d., n=10; FIGS. 9K-9N), indicating that they are less dynamic than CAG RNA foci. This result suggests a stronger intermolecular interaction between the GGGGCC repeats, which is consistent with intracellular G-quadruplex formation (FIG. 9A).

The GGGGCC RNA foci recruited hnRNP H, as previously shown (see, e.g., Conlon et al., eLife 5:345, 2016), as well as MBNL1, and co-localized with nuclear speckles (FIG. 10). Like the 47× CAG RNA, most GGGGCC RNA was retained in the nucleus (about 60%; FIG. 11A). The c9orf72 GGGGCC expansion is located in an intron with about 150 bases upstream and about 6 kb region downstream of the repeats. It was found that the incorporation of the endogenous (about 150 bases) upstream or a long (about 1.7 kb) downstream flanking sequence did not affect the formation of RNA foci (FIG. 11B). Intriguingly, incorporation of a longer sequence (about 1 kb) upstream of the repeats abolished the formation of nuclear puncta (FIG. 11B), suggesting that sequences flanking the repeats may influence their assembly into RNA foci. Similar to the CAG RNA foci, the GGGGCC RNA foci were disrupted by antisense oligonucleotides, doxorubicin, or ammonium acetate (FIGS. 11C-11G), indicating that both base-pairing and electrostatic interactions are essential for GGGGCC RNA foci formation.

In summary, the examples demonstrated that the propensity of an RNA to form multivalent base-pairing can lead to its gelation without requiring protein components. The results showed that sequence-specific base-pairing properties of RNAs can lead to their phase separation and gelation, and raise the possibility that such phenomena could contribute to physiological granule assembly as well. In the case of repeat expansions diseases, the data suggest that intermolecular base-pairing can result in the aggregation and sequestration of RNA into nuclear foci (see, e.g., FIG. 12). RNA gelation, which occurs at a boundary condition of increasing valency, might explain why disease appears to be triggered after an expansion of a nucleotide repeat has reached a threshold number (FIG. 12). The results may also explain why placement of distinct repeat expansions in seemingly unrelated genes can result in similar clinical syndromes (see, e.g., Holmes et al., Nat. Genet. 29:377, 2001 and Elden et al., Nature 466:1069, 2010).

It is understood that the embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An isolated cell comprising:
a heterologous polynucleotide comprising a promoter operably linked to a polynucleotide for encoding an RNA transcript comprising (i) an RNA sequence comprising tandem nucleotide repeats and (ii) a binding motif for binding to a detectable molecule; and
a heterologous detectable molecule that binds to the binding motif.

2. The isolated cell of claim 1 comprising an RNA transcript encoded by the heterologous polynucleotide, wherein the RNA transcript comprises (i) tandem nucleotide repeats and (ii) a binding motif for binding to a detectable molecule; and a heterologous detectable molecule that binds to the binding motif.

3. The isolated cell of claim 1, wherein the tandem nucleotide repeats are trinucleotide repeats selected from CAG repeats, CGG repeats, GCC repeats, GAA repeats, and CUG repeats.

4. (canceled)

5. The isolated cell of claim 3, wherein the RNA sequence comprises at least 30 repeats.

6. The isolated cell of claim 1, wherein the tandem nucleotide repeats are tetranucleotide repeats, pentanucleotide repeats, or hexanucleotide repeats.

7. The isolated cell of claim 6, wherein the tandem nucleotide repeat sequences are GGGGCC repeats, CCUG repeats, or AUUCU repeats.

8. The isolated cell of claim 7, wherein the RNA sequence comprises at least 15 repeats.

9. The isolated cell of claim 1, wherein the binding motif comprises a hairpin loop sequence comprising a plurality of hairpin loop nucleotide sequences separated by a spacer sequence or an aptamer sequence, and the detectable molecule is a heterologous protein that comprises a detectable label selected from a fluorophore or a fluorescent protein.

10-12. (canceled)

13. The isolated cell of claim 9, wherein the hairpin loop sequence comprises a plurality of MS2 hairpin loops, and wherein the detectable molecule comprises an MS2 coat binding protein (MCP).

14. The isolated cell of claim 9, wherein the hairpin loop sequence comprises a PP7 hairpin sequence, and wherein the detectable molecule comprises a PP7 coat binding protein.

15. The isolated cell of claim 2, wherein the binding motif comprises a hairpin loop sequence or an aptamer sequence, and wherein the detectable molecule comprises a U1A RNA-binding protein.

16. The isolated cell of claim 2, wherein the binding motif comprises an RNA aptamer sequence and wherein the detectable molecule is a fluorogen.

17. The isolated cell of claim 16, wherein the RNA aptamer is a Spinach aptamer or a variant or derivative thereof

18. The isolated cell of claim 1, wherein the promoter is an inducible promoter.

19. (canceled)

20. The isolated cell of claim 2, wherein the cell is a mammalian cell.

21. (canceled)

22. A method of detecting the formation of cellular clusters of RNA, the method comprising:
(a) inducing transcription of the RNA sequence in the cell of claim 2, thereby forming transcribed RNAs comprising a sequence that is prone to forming clusters of RNA; and
(b) detecting in the cell the formation of one or more clusters of RNA.

23. (canceled)

24. The method of claim 22, wherein the detecting step (b) comprises detecting the formation of one or more clusters of RNA in the nucleus of the cell.

25. A method of identifying an agent that dissolves or inhibits the formation of cellular clusters of RNA, the method comprising:
(a) contacting an agent to the cell of claim 2, wherein the cell comprises a plurality of RNA transcripts forming clusters of RNA;
(b) quantifying the amount of clusters of RNA formed by the RNA transcripts in the cell that has been contacted with the agent; and
(c) comparing the amount of clusters of RNA formed in (b) with a control value, wherein an amount of clusters of RNA formed in (b) that is less than the control value identifies the agent as an agent that dissolves or inhibits the formation of the clusters of RNA.

26. The method of claim 25, wherein the control value is an amount of clusters of RNA formed by the RNA transcripts in the cell prior to the contacting step (b).

27. The method of claim 25, wherein the method comprises quantifying the amount of clusters of RNA formed in the nucleus of the cell.

28. The method of claim 25, wherein the agent is a small molecule, an oligonucleotide, a nucleic acid intercalator, or a protein.

29-37. (canceled)

38. The isolated cell of claim 2, wherein the tandem repeats are contiguous or non-contiguous.


SEQUENCE LISTING (30 sequences)

SEQ ID NO: 1 - RNA, 141 nt, Artificial Sequence
  Description of Artificial Sequence Synthetic polynucleotide
cugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcug

SEQ ID NO: 2 - RNA, 141 nt, Artificial Sequence
  Description of Artificial Sequence Synthetic polynucleotide
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 3 - RNA, 198 nt, Artificial Sequence
  Description of Artificial Sequence Synthetic polynucleotide
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 4 - DNA, 18 bp, Artificial Sequence
  Description of Artificial Sequence Synthetic oligonucleotide
ctgctgctgctgctgctg

SEQ ID NO: 5 - DNA, 90 bp, Artificial Sequence
  Description of Artificial Sequence Synthetic oligonucleotide
atatatatatatatatatatatatatatatatatatatatatatatatatatatatatatatatatatatatatatatatatatatatat

SEQ ID NO: 6 - RNA, 15 nt, Artificial Sequence
  Description of Artificial Sequence Synthetic oligonucleotide
cagcagcagcagcag

SEQ ID NO: 7 - RNA, 360 nt, Artificial Sequence
  Description of Artificial Sequence Synthetic polynucleotide
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 8 - RNA, 174 nt, Artificial Sequence
  Description of Artificial Sequence Synthetic polynucleotide
ggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 9 - DNA, 24 bp, Artificial Sequence
  Description of Artificial Sequence Synthetic oligonucleotide
ctgctgctgctgctgctgctgctg

SEQ ID NO: 10 - RNA, 24 nt, Artificial Sequence
  Description of Artificial Sequence Synthetic oligonucleotide
cagcagcagcagcagcagcagcag

SEQ ID NO: 11 - RNA, 138 nt, Artificial Sequence
  Description of Artificial Sequence Synthetic polynucleotide
ccccggccccggccccggccccggccccggccccggccccggccccggccccggccccggccccggccccggccccggccccggccccggccccggccccggccccggccccggccccggccccggccccggccccgg

SEQ ID NO: 12 - RNA, 138 nt, Artificial Sequence
  Description of Artificial Sequence Synthetic polynucleotide
ggggccggggccggggccggggccggggccggggccggggccggggccggggccggggccggggccggggccggggccggggccggggccggggccggggccggggccggggccggggccggggccggggccggggcc

SEQ ID NO: 13 - RNA, 18 nt, Artificial Sequence
  Description of Artificial Sequence Synthetic oligonucleotide
ggggccggggccggggcc

SEQ ID NO: 14 - RNA, 174 nt, Artificial Sequence
  Description of Artificial Sequence Synthetic polynucleotide
ccccggccccggccccggccccggccccggccccggccccggccccggccccggccccggccccggccccggccccggccccggccccggccccggccccggccccggccccggccccggccccggccccggccccggccccggccccggccccggccccggccccggccccgg

SEQ ID NO: 15 - RNA, 54 nt, Artificial Sequence
  Description of Artificial Sequence Synthetic oligonucleotide
ggggccggggccggggccggggccggggccggggccggggccggggccggggcc

SEQ ID NO: 16 - RNA, 96 nt, Artificial Sequence
  Description of Artificial Sequence Synthetic oligonucleotide
ggggccggggccggggccggggccggggccggggccggggccggggccggggccggggccggggccggggccggggccggggccggggccggggcc

SEQ ID NO: 17 - RNA, 18 nt, Artificial Sequence
  Description of Artificial Sequence Synthetic oligonucleotide
ccccggccccggccccgg

SEQ ID NO: 18 - protein, 6 aa, Artificial Sequence
  Description of Artificial Sequence Synthetic 6xhis tag
HHHHHH

SEQ ID NO: 19 - RNA, 30 nt, Artificial Sequence
  Description of Artificial Sequence Synthetic oligonucleotide
cagcagcagcagcagcagcagcagcagcag

SEQ ID NO: 20 - RNA, 60 nt, Artificial Sequence
  Description of Artificial Sequence Synthetic oligonucleotide
cagcagcagcagcagcagcagcagcagcagcagcagcagcagcagcagcagcagcagcag

SEQ ID NO: 21 - RNA, 30 nt, Artificial Sequence
  Description of Artificial Sequence Synthetic oligonucleotide
cugcugcugcugcugcugcugcugcugcug

SEQ ID NO: 22 - RNA, 60 nt, Artificial Sequence
  Description of Artificial Sequence Synthetic oligonucleotide
cugcugcugcugcugcugcugcugcugcugcugcugcugcugcugcugcugcugcugcug

SEQ ID NO: 23 - RNA, 30 nt, Artificial Sequence
  Description of Artificial Sequence Synthetic oligonucleotide
ggggccggggccggggccggggccggggcc

SEQ ID NO: 24 - RNA, 93 nt, Artificial Sequence
  Description of Artificial Sequence Synthetic oligonucleotide
cagcagcagcagcagcagcagcagcagcagcagcagcagcagcagcagcagcagcagcagcagcagcagcagcagcagcagcagcagcagcag

SEQ ID NO: 25 - RNA, 93 nt, Artificial Sequence
  Description of Artificial Sequence Synthetic oligonucleotide
cugcugcugcugcugcugcugcugcugcugcugcugcugcugcugcugcugcugcugcugcugcugcugcugcugcugcugcugcugcugcug

SEQ ID NO: 26 - RNA, 198 nt, Artificial Sequence
  Description of Artificial Sequence Synthetic polynucleotide
cugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcugcug

SEQ ID NO: 27 - RNA, 174 nt, Artificial Sequence; identical to SEQ ID NO: 8
  Description of Artificial Sequence Synthetic polynucleotide
  misc_feature (1)..(174): This sequence may encompass 16-29 “ggggcc” repeating units

SEQ ID NO: 28 - RNA, 16 nt, Artificial Sequence
  Description of Artificial Sequence Synthetic oligonucleotide
cagcagcagccagcag

SEQ ID NO: 29 - RNA, 14 nt, Artificial Sequence
  Description of Artificial Sequence Synthetic oligonucleotide
gcagcagcagcagc

SEQ ID NO: 30 - RNA, 60 nt, Artificial Sequence
  Description of Artificial Sequence Synthetic oligonucleotide
ggggccggggccggggccggggccggggccggggccggggccggggccggggccggggcc